(19) United States
(12) Patent Application Publication
(10) Pub. No.: US 2026/0226085 A1
(43) Pub. Date: Aug. 6, 2026

(54) HETEROCYCLIC COMPOUNDS AS WRN INHIBITORS

(71) Applicant: Synnovation Therapeutics, Inc., Wilmington, DE (US)

(72) Inventors: Yu Bai, Wilmington, DE (US); Jeffrey Yang, Wilmington, DE (US); Chao Qi, Wilmington, DE (US); Liangxing Wu, Wilmington, DE (US); Wenqing Yao, Wilmington, DE (US)

(21) Appl. No.: 19/149,331

(22) PCT Filed: Jan. 19, 2024
(86) PCT No.: PCT/US2024/012151

§ 371 Date: Jul. 18, 2025

Related U.S. Application Data
(60) Provisional application No. 63/440,152, filed on Jan. 20, 2023.
(60) Provisional application No. 63/452,975, filed on Mar. 17, 2023.
(60) Provisional application No. 63/455,791, filed on Mar. 30, 2023.

(51) Int. Cl. C07F 9/6561; A61K 31/519; A61K 31/675; A61P 35/00; C07D 487/04; C07D 519/00

(52) U.S. Cl. CPC C07F 9/6561; A61K 31/519; A61K 31/675; A61P 35/00; C07D 487/04; C07D 519/00

(57) ABSTRACT

The present application provides heterocyclic compounds that modulate the activity of Werner syndrome helicase (WRN), and are useful in the treatment of various diseases, including cancer.

### TECHNICAL FIELD

The present disclosure provides heterocyclic compounds as well as their pharmaceutical compositions that modulate the activity of Werner syndrome helicase (WRN) and are useful in the treatment of various diseases related to WRN.

### BACKGROUND

Genome instability is a hallmark of cancer cells. Defective repair of DNA lesions arising from normal cellular processes such as replication or external insults strongly predisposes cells to malignant transformation. One mechanism involved in the maintenance of genome stability is DNA mismatch repair (MMR) that identifies and corrects errors in DNA base pair mismatches or small insertion or deletions.

Germline or somatic mutations in genes encoding MMR proteins or their silencing is associated with high genome instability particularly in regions of highly repetitive DNA, such as TA repeats, also called microsatellites. The phenomenon characterized by expansion of these repeats resulting from defective MMR is termed Microsatellite Instability (MSI). Subsets of many cancer types including colon, ovarian, endometrial and gastric exhibit MSI-high (MSI-H) or defective MMR (dMMR) and can be detected clinically using appropriate diagnostic techniques (Bonneville et al. JCO Precision Oncology, 2017, 1, 1-15).

The Werner Syndrome gene WRN encodes a multi-functional protein of the RECQ family of DNA helicases that also harbors nuclease domain. WRN catalyzes the unwinding and removal of abnormal DNA structures thereby promoting repair of damaged DNA. Analyses of functional genomic CRISPR screens revealed that cancer cell lines characterized by MSI were selectively inhibited by depletion of WRN compared with MS stable (MSS) cell lines indicating a synthetic lethal interaction between WRN and MSI cell lines. Loss of WRN was associated with increased signs of DNA damage that induced cell cycle arrest and apoptosis (Behan et al. Nature, 2019, 568, 511-516; Chan et al. Nature, 2019, 568, 551-556; Lieb et al. Elife, 2019, 8: e43333). The helicase/ATPase activity of WRN was shown to be essential for the ability of WRN to rescue the lethal WRN knockdown phenotype (Kategaya et al. iScience, 2019, 13, 488-497). These reconstitution experiments are consistent with data showing that the WRN helicase is required for unwinding abnormal secondary structures formed from large-scale TA repeats in MSI cell lines (Wietmarschen et al. Nature, 2020, 586, 292-298). In the absence of WRN helicase activity, these repeats are susceptible to attack by nucleases that can result in chromosome shattering leading to cell death. Therefore, targeting WRN, and specifically inhibiting the helicase/ATPase function, could have therapeutic benefit in tumors characterized by MSI-High or dMMR.

### SUMMARY

The present disclosure provides, inter alia, compounds of Formula I.

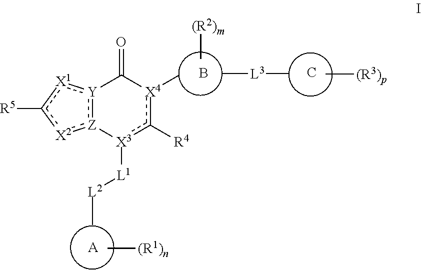

or pharmaceutically acceptable salts thereof, wherein constituent members are defined herein.

The present disclosure further provides a pharmaceutical composition comprising a compound of the disclosure, or a pharmaceutically acceptable salt thereof, and a pharmaceutically acceptable carrier or excipient.

The present disclosure further provides methods of inhibiting WRN activity, comprising contacting the WRN with a compound described herein, or a pharmaceutically acceptable salt thereof.

The present disclosure further provides methods of treating a disease or a disorder associated with WRN in a patient by administering to the patient a therapeutically effective amount of a compound of the disclosure, or a pharmaceutically acceptable salt thereof.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DETAILED DESCRIPTION

The present disclosure provides a compound of Formula I:

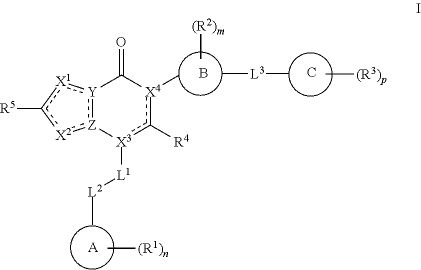

or a pharmaceutically acceptable salt thereof, wherein:
X¹ is N, O, S, NR⁶, or CR⁷;
X² is N, O, S, NR⁸, or CR⁹;
X³ is C or N;
X⁴ is C or N;
Y is C or N;
Z is C or N;
each [symbol] is independently a single or double bond;
n is 0, 1, 2, 3, 4, 5, or 6;
m is 0, 1, 2, 3, 4, 5, or 6;
p is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is C₃₋₁₄ cycloalkyl, C₆₋₁₄ aryl, 4-14 membered heterocycloalkyl, or 5-14 membered heteroaryl;
L¹, L², and L³ are each independently selected from bond, C₁₋₆ alkylene, C₁₋₆ haloalkylene, C₃₋₇ cycloalkylene, 4-7 membered heterocycloalkylene, phenylene, 5-6 membered heteroarylene, —C₃₋₇ cycloalkylene-C₁₋₄ alkyl-, -(4-7 membered heterocycloalkylene)-C₁₋₄ alkyl-, -phenylene-C₁₋₄ alkyl-, -(5-6 membered heteroarylene)-C₁₋₄ alkyl-, —O—, —N(R^{L})—, —C(O)—, —N(R^{L})C(O)—, —N(R^{L})C(O)N(R^{L})—, —N(R^{L})C(O)O—, —S(O)—, —S(O)₂—, —S(O)(═NR^{L})—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, phenylene, 5-6 membered heteroarylene, C₃₋₇ cycloalkylene-C₁₋₄ alkyl, (4-7 membered heterocycloalkylene)-C₁₋₄ alkyl, -phenylene-C₁₋₄ alkyl-, and (5-6 membered heteroarylene)-C₁₋₄ alkyl of L¹, L², and L³ are each optionally substituted with 1, 2, 3, or 4 independently selected R^{G} substituents;
provided that when L³ is —C(O)—, —S(O)—, —S(O)₂—, or —S(O)(═NR^{L})—, then Ring C is C₉₋₁₄ cycloalkyl, C₁₀₋₁₄ aryl, 8-14 membered heterocycloalkyl, or 9-14 membered heteroaryl;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —NO₂, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R¹ are each optionally substituted with 1, 2, 3, 4, 5, or 6 independently selected RA substituents;
or, two R¹ together with the atoms to which they are attached form a C₃₋₇ cycloalkyl, phenyl, 4-7 membered heterocycloalkyl, or 5-6 membered heteroaryl group, wherein the C₃₋₇ cycloalkyl, phenyl, 4-7 membered heterocycloalkyl, and 5-6 membered heteroaryl group is optionally substituted with 1, 2, 3, 4, 5, or 6 independently selected RA substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1}, R^{c1}, and R^{d1} are each optionally substituted with 1, 2, 3, 4, 5, or 6 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected RA substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b1} are each optionally substituted with 1, 2, 3, 4, 5, or 6 independently selected RA substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{1A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1A}, —SR^{a1A}, —NR^{c1A}R^{d1A}, —NO₂, —C(O)R^{a1A}, —C(O)OR^{a1A}, —C(O)NR^{c1A}R^{d1A}, —C(O)NR^{c1A}(OR^{a1A}), —OC(O)R^{a1A}, —OC(O)NR^{c1A}R^{d1A}, —OC(O)OR^{a1A}, —OS(O)₂R^{b1A}, —OS(O)₂NR^{c1A}R^{d1A}, —NR^{c1A}C(O)R^{a1A}, —NR^{c1A}C(O)OR^{a1A}, —NR^{c1A}C(O)NR^{c1A}R^{d1A}, —NR^{c1A}S(O)₂R^{b1A}, —NR^{clA}S(O)₂NR^{c1A}R^{d1A}, —NR^{c1A}OR^{a1A}, —NR^{c1A}S(O)R^{b1A}, —NR^{c1A}S(O)NR^{c1A}R^{d1A}, —S(O)R^{b1A}, —S(O)₂R^{b1A}, —S(O)NR^{c1A}R^{d1A}, —S(O)₂NR^{c1A}R^{d1A}, —C(═NR^{e1A})R^{a1A}, —C(═NR^{e1A})R^{c1A}R^{d1A}, —NR^{c1A}C(═NR^{e1A})R^{a1A}, —NR^{c1A}C(═NR^{e1A})NR^{c1A}R^{d1A}, —NR^{c1A}S(O)(═NR^{e1A})R^{b1A}, —NR^{c1A}S(O)(═NR^{e1A})NR^{c1A}R^{d1A}, —OS(O)(═NR^{e1A})R^{b1A}, —S(O)(═NR^{e1A})R^{b1A}, —S(O)(═NR^{e1A})R^{c1A}R^{d1A}, —C(O)NR^{c1A}S(O)₂R^{b1A}, —C(O)NR^{c1A}S(O)₂NR^{c1A}R^{d1A}, —S(O)₂NR^{c1A}C(O)R^{b1A}, —NR^{c1A}S(O)NR^{c1A}C(O)R^{b1A}, and —P(O)R^{f1A}R^{g1A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{1A} are each optionally substituted with 1, 2, 3, 4, 5, or 6 independently selected R^{G} substituents;
each R^{a1A}, R^{c1A} and R^{d1A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1A}, R^{c1A}, and R^{d1A} are each optionally substituted with 1, 2, 3, 4, 5, or 6 independently selected R^{G} substituents;
or, any R^{c1A} and R^{d1A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b1A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b1A} are each optionally substituted with 1, 2, 3, 4, 5, or 6 independently selected R^{G} substituents;
each R^{e1A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1A} and R^{g1A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —S(O)(═NR^{e2})R^{b2}, and —S(O)(═NR^{e2})NR^{c2}R^{d2}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R² are each optionally substituted with 1, 2, 3, 4, 5, or 6 independently selected R^{G} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a2}, R^{b2}, R^{c2}, and R^{d2} are each optionally substituted with 1, 2, 3, 4, 5, or 6 independently selected R^{G} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R³ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3}, —SR^{a3}, —NR^{c3}R^{d3}, —NO₂, —C(O)R^{a3}, —C(O)OR^{a3}, —C(O)NR^{c3}R^{d3}, —C(O)NR^{c3}(OR^{a3}), —OC(O)R^{a3}, —OC(O)NR^{c3}R^{d3}, —OC(O)OR^{a3}, —OS(O)₂R^{b3}, —OS(O)₂NR^{c3}R^{d3}, —NR^{c3}C(O)R^{a3}, —NR^{c3}C(O)OR^{a3}, —NR^{c3}C(O)NR^{c3}R^{d3}, —NR^{c3}S(O)₂R^{b3}, —NR^{c3}S(O)₂NR^{c3}R^{d3}, —NR^{c3}OR^{a3}, —NR^{c3}S(O)R^{b3}, —NR^{c3}S(O)NR^{c3}R^{d3}, —S(O)R^{b3}, —S(O)₂R^{b3}, —S(O)NR^{c3}R^{d3}, —S(O)₂NR^{c3}R^{d3}, —C(═NR^{e3})R^{a3}, —C(═NR^{e3})NR^{c3}R^{d3}, —NR^{c3}C(═NR^{e3})R^{a3}, —NR^{c3}C(═NR^{e3})NR^{c3}R^{d3}, —NR^{c3}S(O)(═NR^{e3})R^{b3}, —NR^{c3}S(O)(═NR^{e3})NR^{c3}R^{d3}, —OS(O)(═NR^{e3})R^{b3}, —S(O)(═NR^{e3})R^{b3}, —S(O)(═NR^{e3})NR^{c3}R^{d3}, —C(O)NR^{c3}S(O)₂R^{b3}, —C(O)NR^{c3}S(O)₂NR^{c3}R^{d3}, —S(O)₂NR^{c3}C(O)R^{b3}, —NR^{c3}S(O)NR^{c3}C(O)R^{b3}, and —P(O)R^{f3}R^{g3}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R³ are each optionally substituted with 1, 2, 3, 4, 5, or 6 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a3}, R^{c3}, and R^{d3} are each optionally substituted with 1, 2, 3, 4, 5, or 6 independently selected R^{3A} substituents;
or, any R^{c3} and R^{d3} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{3A} substituents;
each R^{b3} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b3} are each optionally substituted with 1, 2, 3, 4, 5, or 6 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{3A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3A}, —SR^{a3A}, —NR^{c3A}R^{d3A}, —NO₂, —C(O)R^{a3A}, —C(O)OR^{a3A}, —C(O)NR^{c3A}R^{d3A}, —C(O)NR^{c3A}(OR^{a3A}), —OC(O)R^{a3A}, —OC(O)NR^{c3A}R^{d3A}, —OC(O)OR^{a3A}, —OS(O)₂R^{b3A}, —OS(O)₂NR^{c3A}R^{d3A}, —NR^{c3A}C(O)R^{a3A}, —NR^{c3A}C(O)OR^{a3A}, —NR^{c3A}C(O)NR^{c3A}R^{d3A}, —NR^{c3A}S(O)₂R^{b3A}, —NR^{c3A}S(O)₂NR^{c3A}R^{d3A}, —NR^{c3A}OR^{a3A}, —NR^{c3A}S(O)R^{b3A}, —NR^{c3A}S(O)NR^{c3A}R^{d3A}, —S(O)R^{b3A}, —S(O)₂R^{b3A}, —S(O)NR^{c3A}R^{d3A}, —S(O)₂NR^{c3A}R^{d3A}, —C(═NR^{e3A})R^{a3A}, —C(═NR^{e3A})NR^{c3A}R^{d3A}, —NR^{c3A}C(═NR^{e3A})R^{a3A}, —NR^{c3A}C(═NR^{e3A})NR^{c3A}R^{d3A}, —NR^{c3A}S(O)(═NR^{e3A})R^{b3A}, —NR^{c3A}S(O)(═NR^{e3A})NR^{c3A}R^{d3A}, —OS(O)(═NR^{e3A})R^{b3A}, —S(O)(═NR^{e3A})R^{b3A}, —S(O)(═NR^{e3A})NR^{c3A}R^{d3A}, —C(O)NR^{c3A}S(O)₂R^{b3A}, —C(O)NR^{c3A}S(O)₂NR^{c3A}R^{d3A}, —S(O)₂NR^{c3A}C(O)R^{b3A}, —NR^{c3A}S(O)NR^{c3A}C(O)R^{b3A}, and —P(O)R^{f3A}R^{g3A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{3A} are each optionally substituted with 1, 2, 3, 4, 5, or 6 independently selected R^{G} substituents;
each R^{a3A}, R^{c3A}, and R^{d3A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a3A}, R^{c3A}, and R^{d3A} are each optionally substituted with 1, 2, 3, 4, 5, or 6 independently selected R^{G} substituents;
or, any R^{c3A} and R^{d3A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b3A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b3A} are each optionally substituted with 1, 2, 3, 4, 5, or 6 independently selected R^{G} substituents;
each R^{e3A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3A} and R^{g3A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}, —NR^{c4}R^{d4}, —C(O)R^{a4}, —C(O)OR^{a4}, —C(O)NR^{c4}R^{d4}, —C(O)NR^{c4}(OR^{a4}), —OC(O)NR^{c4}R^{d4}, —NR^{c4}C(O)R^{a4}, —NR⁴C(O)OR^{a4}, —NR^{c4}C(O)NR^{c4}R^{d4}, —NR⁴S(O)₂R^{b4}, —NR^{c4}S(O)₂NR^{c4}R^{d4}, —NR^{c4}OR^{a4}, —NR^{c4}S(O)R^{b4}, —NR^{c4}S(O)NR^{c4}R^{d4}, —S(O)R^{b4}, —S(O)₂R^{b4}, —S(O)NR^{c4}R^{d4}, —S(O)₂NR^{c4}R^{d4}, —C(═NR^{c4})R^{a4}, —C(═NR^{e4})NR^{c4}R^{d4}, —NR^{c4}C(═NR^{e4})R^{a4}, —NR^{c4}C(═NR^{e4})NR^{c4}R^{d4}, —NR^{c4}S(O)(═NR^{e4})R^{b4}, —NR^{c4}S(O)(═NR^{e4})NR^{c4}R^{d4}, —S(O)(═NR^{e4})R^{b4}, and —S(O)(═NR^{e4})NR^{c4}R^{d4}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
or, R⁴ and L¹, together with the atoms to which they are attached, form a C₅₋₁₀ cycloalkyl, phenyl, 5-10 membered heterocycloalkyl, or 5-6 membered heteroaryl group, wherein the C₅₋₁₀ cycloalkyl, phenyl, 5-10 membered heterocycloalkyl, and 5-6 membered heteroaryl group is optionally substituted with 1, 2, 3, 4, 5, or 6 independently selected R^{G} substituents;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a4}, R^{b4}, R^{c4}, and R^{d4} are each optionally substituted with 1, 2, 3, 4, 5, or 6 independently selected R^{G} substituents;
or, any R^{c4} and R^{d4} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, 4, 5, or 6 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5}, R^{c5}, and R^{d5} are each optionally substituted with 1, 2, 3, 4, 5, or 6 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b5} are each optionally substituted with 1, 2, 3, 4, 5, or 6 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{5A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5A}, —SR^{a5A}, —NR^{c5A}R^{d5A}, —NO₂, —C(O)R^{a5A}, —C(O)OR^{a5A}, —C(O)NR^{c5A}R^{d5A}, —C(O)NR^{c5A}(OR^{a5A}), —OC(O)R^{a5A}, —OC(O)NR^{c5A}R^{d5A}, —OC(O)OR^{a5A}, —OS(O)₂R^{b5A}, —OS(O)₂NR^{c5A}R^{d5A}, —NR^{c5A}C(O)R^{a5A}, —NR^{c5A}C(O)OR^{a5A}, —NR^{c5A}C(O)NR^{c5A}R^{d5A}, —NR^{c5A}S(O)₂R^{b5A}, —NR^{c5A}S(O)₂NR^{c5A}R^{d5A}, —NR^{c5A}OR^{a5A}, —NR^{c5A}S(O)R^{b5A}, —NR^{c5A}S(O)NR^{c5A}R^{d5A}, —S(O)R^{b5A}, —S(O)₂R^{b5A}, —S(O)NR^{c5A}R^{d5A}, —S(O)₂NR^{c5A}R^{d5A}, —C(═NR^{5A})R^{a5A}, —C(═NR^{e5A})NR^{c5A}R^{d5A}, —NR^{c5A}C(═NR^{e5A})R^{a5A}, —NR^{c5A}C(═NR^{e5A})NR^{c5A}R^{d5A}, —NR^{c5A}S(O)(═NR^{e5A})R^{b5A}, —NR^{c5A}S(O)(═NR^{e5A})NR^{c5A}R^{d5A}, —OS(O)(═NR^{e5A})R^{b5A}, —S(O)(═NR^{e5A})R^{b5A}, —S(O)(═NR^{e5A})R^{c5A}R^{d5A}, —C(O)NR^{c5A}S(O)₂R^{b5A}, —C(O)NR^{c5A}S(O)₂NR^{c5A}R^{d5A}, —S(O)₂NR^{c5A}C(O)R^{b5A}, —NR^{c5A}S(O)NR^{c5A}C(O)R^{b5A} and —P(O)R^{f5A}R^{g5A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{5A} are each optionally substituted with 1, 2, 3, 4, 5, or 6 independently selected R^{G} substituents;
each R^{a5A}, R^{c5A}, and R^{d5A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5A}, R^{c5A}, and R^{d5A} are each optionally substituted with 1, 2, 3, 4, 5, or 6 independently selected R^{G} substituents;
or, any R^{c5A} and R^{d5A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b5A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b5A} are each optionally substituted with 1, 2, 3, 4, 5, or 6 independently selected R^{G} substituents;
each R^{e5A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5A} and R^{g5A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl;
R⁹ is selected from H, halo, CN, C₁₋₆ alkyl, or C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, X¹ is N.

In some embodiments, X¹ is O.

In some embodiments, X¹ is S.

In some embodiments, X¹ is NR.

In some embodiments, X¹ is CR⁷.

In some embodiments, X² is N.

In some embodiments, X² is O.

In some embodiments, X² is S.

In some embodiments, X² is NR⁸.

In some embodiments, X² is CR⁹.

In some embodiments, X¹ is N and X² is N.

In some embodiments, X³ is C.

In some embodiments, X³ is N.

In some embodiments, X¹ and X³ are each N.

In some embodiments, X² and X³ are each N.

In some embodiments, X¹, X², and X³ are each N.

In some embodiments, X⁴ is C.

In some embodiments, X⁴ is N.

In some embodiments, X¹, X², and X³ are each N and X⁴ is C.

In some embodiments, Y is C.

In some embodiments, Y is N.

In some embodiments, Z is C.

In some embodiments, Z is N.

In some embodiments, Y is N and Z is C.

In some embodiments:
X¹, X², and X³ are each N;
X⁴ is C;
Y is N; and
Z is C.

In some embodiments, L¹ is selected from a bond, C₁₋₆ alkylene, and C₁₋₆ haloalkylene.

In some embodiments, L¹ is C₁₋₆ alkylene.

In some embodiments, L¹ is C₁₋₃ alkylene.

In some embodiments, L¹ is —CH₂— (i.e., L¹ is methylene).

In some embodiments, L² is selected from —N(R^{L})—, —C(O)—, and —N(R^{L})C(O)—.

In some embodiments, L² is —N(R^{L})C(O)—.

In some embodiments, each R^{L} is independently selected from H and C₁₋₆ alkyl.

In some embodiments, each R^{L} is independently selected from H and C₁₋₃ alkyl.

In some embodiments, each R^{L} is H.

In some embodiments, L² is —NHC(O)—.

In some embodiments, L¹-L² forms —CH₂C(O)NH—.

In some embodiments, L³ is C₁₋₆ alkylene, —C(O)—, —S(O)—, —S(O)₂—, or —S(O)(═NR^{L})—. In some embodiments, L³ is C₁₋₃ alkylene, —C(O)—, —S(O)—, —S(O)₂—, or —S(O)(═NR^{L})—.

In some embodiments, L³ is —C(O)—, —S(O)—, —S(O)₂—, or —S(O)(═NR^{L}).

In some embodiments, L³ is C₁₋₆ alkylene or —C(O)—.

In some embodiments, L³ is C₁₋₃ alkylene or —C(O)—.

In some embodiments, L³ is —CH₂— or —C(O)—.

In some embodiments, L³ is —C(O)—.

In some embodiments, Ring A is C₃₋₇ cycloalkyl, phenyl, 4-7 membered heterocycloalkyl, or 5-6 membered heteroaryl.

In some embodiments, Ring A is C₆₋₁₀ aryl.

In some embodiments, Ring A is phenyl.

In some embodiments, n is 0, 1, 2, 3, or 4.

In some embodiments, n is 1, 2, or 3.

In some embodiments, n is 1 or 2.

In some embodiments, n is 1.

In some embodiments, n is 2.

In some embodiments, each R¹ is independently selected from halo, C₁₋₆ alkyl, C₂₋₆ alkenyl, C₂₋₆ alkynyl, and C₁₋₆ haloalkyl.

In some embodiments, each R¹ is independently selected from halo, C₁₋₆ alkyl, and C₁₋₆ haloalkyl.

In some embodiments, each R¹ is independently selected from halo, C₁₋₃ alkyl, and C₁₋₃ haloalkyl.

In some embodiments, each R¹ is independently selected from halo and C₁₋₆ haloalkyl.

In some embodiments, each R¹ is independently selected from halo and C₁₋₃ haloalkyl.

In some embodiments, each R¹ is independently selected from chloro and trifluoromethyl.

In some embodiments, Ring A is chloro(trifluoromethyl)phenyl.

In some embodiments, Ring A is

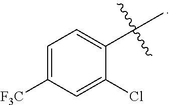 [SMILES: *c1ccc(C(F)(F)F)cc1Cl]

In some embodiments, Ring B is C₃₋₇ cycloalkyl, phenyl, 4-7 membered heterocycloalkyl, or 5-6 membered heteroaryl.

In some embodiments, Ring B is 4-14 membered heterocycloalkyl.

In some embodiments, Ring B is 4-7 membered heterocycloalkyl.

In some embodiments, Ring B is piperazinyl.

In some embodiments, Ring B is

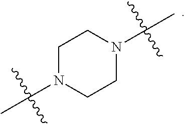 [SMILES: *N1CCN(*)CC1]

In some embodiments, m is 0, 1, 2, 3, or 4.

In some embodiments, m is 0, 1, or 2.

In some embodiments, m is 0 or 1.

In some embodiments, m is 0.

In some embodiments, Ring C is C₉₋₁₄ cycloalkyl, C₁₀₋₁₄ aryl, 8-14 membered heterocycloalkyl, or 5-14 membered heteroaryl.

In some embodiments, Ring C is C₉₋₁₄ cycloalkyl, C₁₀₋₁₄ aryl, 8-14 membered heterocycloalkyl, 5-6 membered heteroaryl, or 9-14 membered heteroaryl.

In some embodiments, Ring C is C₉₋₁₄ cycloalkyl, C₁₀₋₁₄ aryl, 8-14 membered heterocycloalkyl, monocyclic 5-6 membered heteroaryl, or bicyclic 9-14 membered heteroaryl.

In some embodiments, Ring C is C₉₋₁₄ cycloalkyl, C₁₀₋₁₄ aryl, 8-14 membered heterocycloalkyl, or 9-14 membered heteroaryl.

In some embodiments, Ring C is 8-14 membered heterocycloalkyl, 5-6 membered heteroaryl, or 9-14 membered heteroaryl.

In some embodiments, Ring C is 8-14 membered heterocycloalkyl or 9-14 membered heteroaryl.

In some embodiments, Ring C is a bicyclic 8-14 membered heterocycloalkyl or bicyclic 9-14 membered heteroaryl.

In some embodiments, Ring C is 9-14 membered heteroaryl.

In some embodiments, Ring C is a bicyclic 9-14 membered heteroaryl.

In some embodiments, Ring C is pyridinyl, 5H-pyrrolo[3,2-d]pyrimidinyl, quinolinyl, or isoquinolinyl.

In some embodiments, Ring C is 5H-pyrrolo[3,2-d]pyrimidinyl, quinolinyl, or isoquinolinyl.

In some embodiments, Ring C is 5H-pyrrolo[3,2-d]pyrimidinyl.

In some embodiments, Ring C is quinolinyl.

In some embodiments, Ring C is isoquinolinyl.

In some embodiments, Ring C is 5-6 membered heteroaryl.

In some embodiments, L³ is C₁₋₆ alkylene and Ring C is 5-6 membered heteroaryl.

In some embodiments, L³ is C₁₋₃ alkylene and Ring C is 5-6 membered heteroaryl.

In some embodiments, L³ is —CH₂— and Ring C is 5-6 membered heteroaryl.

In some embodiments, Ring C is pyridinyl.

In some embodiments, L³ is C₁₋₆ alkylene and Ring C is pyridinyl.

In some embodiments, L³ is C₁₋₃ alkylene and Ring C is pyridinyl.

In some embodiments, L³ is —CH₂— and Ring C is pyridinyl.

In some embodiments, p is 0, 1, 2, 3, or 4.

In some embodiments, p is 1, 2, 3, or 4.

In some embodiments, p is 0, 1, 2, or 3.

In some embodiments, p is 0, 1, or 2.

In some embodiments, p is 0 or 1.

In some embodiments, p is 1.

In some embodiments, p is 0.

In some embodiments, each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3}.

In some embodiments, each R^{a3}, R^{b3}, R^{c3}, and R^{d3} is independently selected from H, C₁₋₆ alkyl, and C₁₋₆ haloalkyl.

In some embodiments, each R^{a3}, R^{b3}, R^{c3}, and R^{d3} is independently selected from H and C₁₋₆ alkyl.

In some embodiments, each R^{a3}, R^{c3}, and R^{d3} is independently selected from H, C₁₋₆ alkyl, and C₁₋₆ haloalkyl.

In some embodiments, each R^{a3}, R^{c3}, and R^{d3} is independently selected from H and C₁₋₆ alkyl.

In some embodiments, each R^{a3} is independently selected from H, C₁₋₆ alkyl, and C₁₋₆ haloalkyl.

In some embodiments, each R^{a3} is independently selected from H and C₁₋₆ alkyl.

In some embodiments, each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3}; and each R^{a3}, R^{c3}, and R^{d3} is independently selected from H, C₁₋₆ alkyl, and C₁₋₆ haloalkyl.

In some embodiments, each R³ is independently selected from —OR^{a3}; and each R^{a3}, R^{c3}, and R^{d3} is independently selected from H and C₁₋₆ alkyl.

In some embodiments, each R³ is hydroxy.

In some embodiments, R⁴ is selected from H, halo, C₁₋₆ alkyl, C₂₋₆ alkenyl, C₂₋₆ alkynyl, C₁₋₆ haloalkyl.

In some embodiments, R⁴ is C₁₋₆ alkyl.

In some embodiments, R⁴ is C₁₋₃ alkyl.

In some embodiments, R⁴ is ethyl.

In some embodiments, R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents.

In some embodiments, R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments, R⁵ is selected from C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵ are each optionally substituted with 1, 2, 3, or 4 independently selected R^{5A} substituents.

In some embodiments, R⁵ is selected from C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl.

In some embodiments, R⁵ is selected from C₃₋₁₀ cycloalkyl, C₆₋₁₀ aryl, 4-10 membered heterocycloalkyl, and 5-10 membered heteroaryl, wherein the C₃₋₁₀ cycloalkyl, C₆₋₁₀ aryl, 4-10 membered heterocycloalkyl, and 5-10 membered heteroaryl of R⁵ are each optionally substituted with 1, 2, 3, or 4 independently selected R^{5A} substituents.

In some embodiments, R⁵ is selected from C₃₋₁₀ cycloalkyl, C₆₋₁₀ aryl, 4-10 membered heterocycloalkyl, and 5-10 membered heteroaryl.

In some embodiments, R⁵ is selected from C₃₋₇ cycloalkyl, phenyl, 4-7 membered heterocycloalkyl, and 5-6 membered heteroaryl, wherein the C₃₋₇ cycloalkyl, phenyl, 4-7 membered heterocycloalkyl, and 5-6 membered heteroaryl of R⁵ are each optionally substituted with 1, 2, 3, or 4 independently selected R^{5A} substituents.

In some embodiments, R⁵ is selected from C₃₋₇ cycloalkyl, phenyl, 4-7 membered heterocycloalkyl, and 5-6 membered heteroaryl.

In some embodiments, R⁵ is C₆₋₁₀ aryl, which is optionally substituted with 1, 2, 3, or 4 independently selected R^{5A} substituents.

In some embodiments, R⁵ is phenyl, which is optionally substituted with 1, 2, 3, or 4 independently selected R^{5A} substituents.

In some embodiments, R⁵ is phenyl, which is optionally substituted with 1 or 2 independently selected R^{5A} substituents.

In some embodiments, R⁵ is phenyl or 4-10 membered heterocycloalkyl, wherein the phenyl and 4-10 membered heterocycloalkyl of R⁵ are each optionally substituted with 1, 2, 3, or 4 independently selected R^{5A} substituents;
each R^{5A} is independently selected from —S(O)R^{b5A}, —S(O)₂R^{b5A}, —S(O)NR^{c5A}R^{d5A}, —S(O)₂NR^{c5A}R^{d5A} and —P(O)R^{f5A}R^{g5A}; and
   each R^{a5A}, R^{b5A}, R^{c5A}, R^{d5A}, R^{f5A}, and R^{g5A} is independently selected from H, C₁₋₆ alkyl, C₁₋₆ haloalkyl, C₂₋₆ alkenyl, C₂₋₆ alkynyl.

In some embodiments, R⁵ is phenyl or 4-10 membered heterocycloalkyl, wherein the phenyl and 4-10 membered heterocycloalkyl of R⁵ are each optionally substituted with 1, 2, 3, or 4 independently selected R^{5A} substituents; each R^{5A} is independently selected from —S(O)R^{b5A}, —S(O)₂R^{b5A}, —S(O)NR^{c5A}R^{d5A}, and —S(O)₂NR^{c5A}R^{d5A}; and each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl.

In some embodiments, R⁵ is phenyl, dihydropyranyl, or oxaazaspiro[4.5]decanyl, wherein the phenyl is optionally substituted with methylsulfonyl or dimethylphosphoryl.

In some embodiments, R⁵ is phenyl, dihydropyranyl, or oxaazaspiro[4.5]decanyl, wherein the phenyl is optionally substituted with methylsulfonyl.

In some embodiments, each R⁵ is phenyl, which is optionally substituted with methylsulfonyl.

In some embodiments, each R⁵ is phenyl, which is optionally substituted with dimethylphosphoryl.

In some embodiments, R⁵ is 4-10 membered heterocycloalkyl, which is optionally substituted with 1, 2, 3, or 4 independently selected R^{5A} substituents.

In some embodiments, R⁵ is 4-7 membered heterocycloalkyl, which is optionally substituted with 1, 2, 3, or 4 independently selected R^{5A} substituents.

In some embodiments, R⁵ is 4-10 membered heterocycloalkyl.

In some embodiments, R⁵ is 4-7 membered heterocycloalkyl.

In some embodiments, each R^{5A} is independently selected from —S(O)R^{b5A}, —S(O)₂R^{b5A}, —S(O)NR^{c5A}R^{d5A}, —S(O)₂NR^{c5A}R^{d5A} and —P(O)R^{f5A}R^{g5A}.

In some embodiments, each R^{5A} is independently selected from —S(O)R^{b5A}, —S(O)₂R^{b5A}, —S(O)NR^{c5A}R^{d5A}, and —S(O)₂NR^{c5A}R^{d5A}.

In some embodiments, each R^{5A} is independently selected from —S(O)R^{b5A}S(O)₂R^{b5A}, and —P(O)R^{f5A}R^{g5A}.

In some embodiments, each R^{5A} is independently selected from S(O)₂R^{b5A} and —P(O)R^{f5A}R^{g5A}.

In some embodiments, each R^{5A} is independently selected from —S(O)R^{b5A} and S(O)₂R^{b5A}.

In some embodiments, each R^{5A} is independently selected from S(O)₂R^{b5A}.

In some embodiments, each R^{5A} is independently selected from —P(O)R^{f5A}R^{g5A}.

In some embodiments, each R^{a5A}, R^{b5A}, R^{c5A}, R^{d5A}, R^{f5A}, and R^{g5A} independently selected from H, C₁₋₆ alkyl, C₁₋₆ haloalkyl, C₂₋₆ alkenyl, C₂₋₆ alkynyl.

In some embodiments, each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl.

In some embodiments, each R^{a5A}, R^{b5A}, R^{c5A}, R^{d5A}, R^{f5A}, and R^{g5A} is independently selected from H and C₁₋₆ alkyl.

In some embodiments, each R^{a5A}, R^{b5A}, R^{c5A}, and R^{d5A} is independently selected from H and C₁₋₆ alkyl.

In some embodiments, each R^{a5A}, R^{b5A}, R^{c5A}, R^{d5A}, R^{f5}, and R^{g5} is independently selected from C₁₋₆ alkyl.

In some embodiments, each R^{a5A}, R^{b5A}, R^{c5A}, and R^{d5A} is independently selected from C₁₋₆ alkyl.

In some embodiments, each R^{b5A}, R^{f5A}, and R^{g5A} is independently selected from H, C₁₋₆ alkyl, C₁₋₆ haloalkyl, C₂₋₆ alkenyl, C₂₋₆ alkynyl.

In some embodiments, each R^{b5A} is independently selected from H, C₁₋₆ alkyl, C₁₋₆ haloalkyl, C₂₋₆ alkenyl, C₂₋₆ alkynyl.

In some embodiments, each R^{b5A}, R^{f5A}, and R^{g5A} is independently selected from H and C₁₋₆ alkyl.

In some embodiments, each R^{b5A} is independently selected from H and C₁₋₆ alkyl.

In some embodiments, each R^{b5A}, R^{f5A}, and R^{g5A} is independently selected from C₁₋₆ alkyl.

In some embodiments, each R^{b5A} is independently selected from C₁₋₆ alkyl.

In some embodiments, each R^{5A} is methylsulfonyl or dimethylphosphoryl.

In some embodiments, each R^{5A} is methylsulfonyl.

In some embodiments, each R^{5A} is dimethylphosphoryl.

In some embodiments, R⁵ is selected from phenyl, dihydropyranyl, and oxaazaspiro[4.5]decanyl, wherein the phenyl, dihydropyranyl, and oxaazaspiro[4.5]decanyl are each optionally substituted with 1, 2, 3, or 4 independently selected R^{5A} substituents.

In some embodiments, R⁵ is o

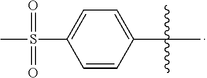 [SMILES: *c1ccc(S(C)(=O)=O)cc1]

In some embodiments, R⁵ is

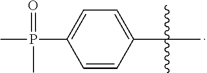 [SMILES: *c1ccc(P(C)(C)=O)cc1]

In embodiments, R⁵ is dihydropyranyl or oxaazaspiro[4.5]decanyl.

In some embodiments, R⁵ is oxaazaspiro[4.5]decanyl.

In some embodiments, R⁵ is 1-oxa-8-azaspiro[4.5]decanyl.

In some embodiments, R⁵ is

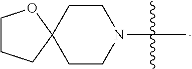 [SMILES: *N1CCC2(CCCO2)CC1]

In some embodiments, R⁵ is dihydropyranyl.

In some embodiments, R⁵ is

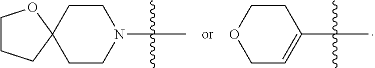 [SMILES: *N1CCC2(CCCO2)CC1 ; *C1=CCOCC1]

In some embodiments, R⁵ is

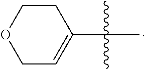 [SMILES: *C1=CCOCC1]

In some embodiments:
X¹ is N, O, S, NR⁶, or CR⁷;
X² is N, O, S, NR⁸, or CR⁹;
X³ is C or N;
X⁴ is C or N;
Y is C or N;
Z is C or N;
each [symbol] is independently a single or double bond;
n is 0, 1, 2, 3, 4, 5, or 6;
m is 0, 1, 2, 3, 4, 5, or 6;
p is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is C₃₋₁₄ cycloalkyl, C₆₋₁₄ aryl, 4-14 membered heterocycloalkyl, or 5-14 membered heteroaryl;
L¹, L², and L³ are each independently selected from bond, C₁₋₆ alkylene, C₁₋₆ haloalkylene, C₃₋₇ cycloalkylene, 4-7 membered heterocycloalkylene, phenylene, 5-6 membered heteroarylene, —C₃₋₇ cycloalkylene-C₁₋₄ alkyl-, -(4-7 membered heterocycloalkylene)-C₁₋₄ alkyl-, -phenylene-C₁₋₄ alkyl-, -(5-6 membered heteroarylene)-C₁₋₄ alkyl-, —O—, —N(R^{L})—, —C(O)—, —N(R^{L})C(O)—, —N(R^{L})C(O)N(R^{L})—, —N(R^{L})C(O)O—, —S(O)—, —S(O)₂—, —S(O)(═NR^{L})—, —S(O)₂N(R^{L})—, and —N(R^{L})S(O)₂N(R^{L})—;
provided that when L³ is —C(O)—, —S(O)—, —S(O)₂—, or —S(O)(═NR^{L})—, then Ring C is C₉₋₁₄ cycloalkyl, C₁₀₋₁₄ aryl, 8-14 membered heterocycloalkyl, or 9-14 membered heteroaryl;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —NO₂, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1};
or, two R¹ together with the atoms to which they are attached form a C₃₋₇ cycloalkyl, phenyl, 4-7 membered heterocycloalkyl, or 5-6 membered heteroaryl group;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c1} and R^{d1} attached to the same N atom, together with the N atom to which they are attached, form a 4-10 membered heterocycloalkyl group;
each R^{b1} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR²C(═NR^{e2})NR^{c2}R^{d2}, —NR^{c2}S(O)(═NR^{e2})R^{b2}, —NR^{c2}S(O)(═NR^{e2})NR^{c2}R^{d2}, —S(O)(═NR^{e2})R^{b2}, and —S(O)(═NR^{e2})R^{c2}R^{d2};
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c2} and R^{d2} attached to the same N atom, together with the N atom to which they are attached, form a 4-7 membered heterocycloalkyl group;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R³ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3}, —SR^{a3}, —NR^{c3}R^{d3}, —NO₂, —C(O)R^{a3}, —C(O)OR^{a3}, —C(O)NR^{c3}R^{d3}, —C(O)NR^{c3}(OR^{a3}), —OC(O)R^{a3}, —OC(O)NR^{c3}R^{d3}, —OC(O)OR^{a3}, —OS(O)₂R^{b3}, —OS(O)₂NR^{c3}R^{d3}, —NR^{c3}C(O)R^{a3}, —NR^{c3}C(O)OR^{a3}, —NR^{c3}C(O)NR^{c3}R^{d3}, —NR³S(O)₂R^{b3}, —NR^{c3}S(O)₂NR^{c3}R^{d3}, —NR^{c3}OR^{a3}, —NR^{c3}S(O)R^{b3}, —NR^{c3}S(O)NR^{c3}R^{d3}, —S(O)R^{b3}, —S(O)₂R¹³, —S(O)NR^{c3}R^{d3}, —S(O)₂NR^{c3}R^{d3}, —C(═NR^{e3})R³, —C(═NR^{e3})NR^{c3}R^{d3}, —NR^{c3}C(═NR^{e3})R^{a3}, —NR^{c3}C(═NR^{e3})NR^{c3}R^{d3}, —NR^{c3}S(O)(═NR^{e3})R^{b3}, —NR^{c3}S(O)(═NR^{e3})NR^{c3}R^{d3}, —OS(O)(═NR^{e3})R^{b3}, —S(O)(═NR^{e3})R^{b3}, —S(O)(═NR^{e3})NR^{c3}R^{d3}, —C(O)NR^{c3}S(O)₂R¹³, —C(O)NR^{c3}S(O)₂NR^{c3}R^{d3}, —S(O)₂NR^{c3}C(O)R^{b3}, —NR^{c3}S(O)NR^{c3}C(O)R^{b3}, and —P(O)R^{f3}R^{g3};
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c3} and R^{d3} attached to the same N atom, together with the N atom to which they are attached, form a 4-10 membered heterocycloalkyl group;
each R^{b3} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}, —NR^{c4}R^{d4}, —C(O)R^{a4}, —C(O)OR^{a4}, —C(O)NR^{c4}R^{d4}, —C(O)NR^{c4}(OR^{a4}), —OC(O)NR^{c4}R^{d4}, —NR^{c4}C(O)R^{a4}, —NR⁴C(O)OR^{a4}, —NR^{c4}C(O)NR^{c4}R^{d4}, —NR⁴S(O)₂R^{b4}, —NR^{c4}S(O)₂NR^{c4}R^{d4}, —NR^{c4}OR^{a4}, —NR^{c4}S(O)R^{b4}, —NR^{c4}S(O)NR^{c4}R^{d4}, —S(O)R^{b4}, —S(O)₂R^{b4}, —S(O)NR^{c4}R^{d4}, —S(O)₂NR^{c4}R^{d4}, —C(═NR^{e4})R^{a4}, —C(═NR^{e4})NR^{c4}R^{d4}, —NR^{c4}C(═NR^{e4})R^{a4}, —NR^{c4}C(═NR^{e4})NR^{c4}R^{d4}, —NR^{c4}S(O)(═NR^{e4})R^{b4}, —NR^{c4}S(O)(═NR^{e4})NR^{c4}R^{d4}, —S(O)(═NR^{e4})R^{b4}, and —S(O)(═NR^{e4})NR^{c4}R^{d4};
or, R⁴ and L¹, together with the atoms to which they are attached, form a C₅₋₁₀ cycloalkyl, phenyl, 5-10 membered heterocycloalkyl, or 5-6 membered heteroaryl group;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c4} and R^{d4} attached to the same N atom, together with the N atom to which they are attached, form a 4-7 membered heterocycloalkyl group;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁-4 alkyl, (4-10 membered heterocycloalkyl)-C₁₋₄ alkyl, and (5-10 membered heteroaryl)-C₁₋₄ alkyl of R⁵ are each optionally substituted with 1, 2, 3, 4, 5, or 6 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁-4 alkyl, (4-10 membered heterocycloalkyl)-C₁₋₄ alkyl, and (5-10 membered heteroaryl)-C₁₋₄ alkyl;
or, any R^{c5} and R^{d5} attached to the same N atom, together with the N atom to which they are attached, form a 4-10 membered heterocycloalkyl group;
each R^{b5} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{5A} is independently selected from —S(O)R^{b5A}, —S(O)₂R^{b5A}, —S(O)NR^{c5A}R^{d5A}, —S(O)₂NR^{c5A}R^{d5A} and —P(O)R^{f5A}R^{g5A};
each R^{a5A}, R^{b5A}, R^{c5A}, R^{d5A}, R^{f5A}, and R^{g5A} is independently selected from H, C₁₋₆ alkyl, C₁₋₆ haloalkyl, C₂₋₆ alkenyl, C₂₋₆ alkynyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

In some embodiments:
X¹ is N, O, S, NR⁶, or CR⁷;
X² is N, O, S, NR⁸, or CR⁹;
X³ is C or N;
X⁴ is C or N;
Y is C or N;
Z is C or N;
each [symbol] is independently a single or double bond;
n is 0, 1, 2, 3, 4, 5, or 6;
m is 0, 1, 2, 3, 4, 5, or 6;
p is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is C₃₋₁₄ cycloalkyl, C₆₋₁₄ aryl, 4-14 membered heterocycloalkyl, or 5-14 membered heteroaryl;
L¹, L², and L³ are each independently selected from bond, C₁₋₆ alkylene, C₁₋₆ haloalkylene, C₃₋₇ cycloalkylene, 4-7 membered heterocycloalkylene, phenylene, 5-6 membered heteroarylene, —C₃₋₇ cycloalkylene-C₁₋₄ alkyl-, -(4-7 membered heterocycloalkylene)-C₁₋₄ alkyl-, -phenylene-C₁₋₄ alkyl-, -(5-6 membered heteroarylene)-C₁₋₄ alkyl-, —O—, —N(R^{L})—, —C(O)—, —N(R^{L})C(O)—, —N(R^{L})C(O)N(R^{L})—, —N(R^{L})C(O)O—, —S(O)—, —S(O)₂—, —S(O)(═NR^{L})—, —S(O)₂N(R^{L})—, and —N(R^{L})S(O)₂N(R^{L})—;
provided that when L³ is —C(O)—, —S(O)—, —S(O)₂—, or —S(O)(═NR^{L})—, then Ring C is C₉₋₁₄ cycloalkyl, C₁₀₋₁₄ aryl, 8-14 membered heterocycloalkyl, or 9-14 membered heteroaryl;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —NO₂, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1};
or, two R¹ together with the atoms to which they are attached form a C₃₋₇ cycloalkyl, phenyl, 4-7 membered heterocycloalkyl, or 5-6 membered heteroaryl group;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c1} and R^{d1} attached to the same N atom, together with the N atom to which they are attached, form a 4-10 membered heterocycloalkyl group;
each R^{b1} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —S(O)(═NR^{e2})R^{b2}, and —S(O)(═NR^{e2})R^{c2}R^{d2};
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c2} and R^{d2} attached to the same N atom, together with the N atom to which they are attached, form a 4-7 membered heterocycloalkyl group;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R³ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3}, —SR^{a3}, —NR^{c3}R^{d3}, —NO₂, —C(O)R^{a3}, —C(O)OR^{a3}, —C(O)NR^{c3}R^{d3}, —C(O)NR^{c3}(OR^{a3}), —OC(O)R^{a3}, —OC(O)NR^{c3}R^{d3}, —OC(O)OR^{a3}, —OS(O)₂R^{b3}, —OS(O)₂NR^{c3}R^{d3}, —NR^{c3}C(O)R^{a3}, —NR^{c3}C(O)OR^{a3}, —NR^{c3}C(O)NR^{c3}R^{d3}, —NR^{c3}S(O)₂R^{b3}, —NR^{c3}S(O)₂NR^{c3}R^{d3}, —NR^{c3}OR^{a3}, —NR^{c3}S(O)R^{b3}, —NR^{c3}S(O)NR^{c3}R^{d3}, —S(O)R^{b3}, —S(O)₂R^{b3}, —S(O)NR^{c3}R^{d3}, —S(O)₂NR^{c3}R^{d3}, —C(═NR^{e3})R^{a3}, —C(═NR^{e3})NR^{c3}R^{d3}, —NR^{c3}C(═NR^{e3})R^{a3}, —NR^{c3}C(═NR^{e3})NR^{c3}R^{d3}, —NR^{c3}S(O)(═NR^{e3})R^{b3}, —NR^{c3}S(O)(═NR^{e3})NR^{c3}R^{d3}, —OS(O)(═NR^{e3})R^{b3}, —S(O)(═NR^{e3})R^{b3}, —S(O)(═NR^{e3})NR^{c3}R^{d3}, —C(O)NR^{c3}S(O)₂R^{b3}, —C(O)NR^{c3}S(O)₂NR^{c3}R^{d3}, —S(O)₂NR^{c3}C(O)R^{b3}, —NR^{c3}S(O)NR^{c3}C(O)R^{b3}, and —P(O)R^{f3}R^{g3};
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c3} and R^{d3} attached to the same N atom, together with the N atom to which they are attached, form a 4-10 membered heterocycloalkyl group;
each R^{b3} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}, —NR^{c4}R^{d4}, —C(O)R^{a4}, —C(O)OR^{a4}, —C(O)NR^{c4}R^{d4}, —C(O)NR^{c4}(OR^{a4}), —OC(O)NR^{c4}R^{d4}, —NR^{c4}C(O)R^{a4}, —NR⁴C(O)OR^{a4}, —NR^{c4}C(O)NR^{c4}R^{d4}, —NR⁴S(O)₂R^{b4}, —NR^{c4}S(O)₂NR^{c4}R^{d4}, —NR^{c4}OR^{a4}, —NR^{c4}S(O)R^{b4}, —NR^{c4}S(O)NR^{c4}R^{d4}, —S(O)R^{b4}, —S(O)₂R¹⁴, —S(O)NR^{c4}R^{d4}, —S(O)₂NR^{c4}R^{d4}, —C(═NR^{e4})R^{a4}, —C(═NR^{e4})NR^{c4}R^{d4}, —NR^{c4}C(═NR^{e4})R^{a4}, —NR^{c4}C(═NR^{e4})NR^{c4}R^{d4}, —NR^{c4}S(O)(═NR^{e4})R^{b4}, —NR^{c4}S(O)(═NR^{e4})NR^{c4}R^{d4}, —S(O)(═NR^{e4})R^{b4}, and —S(O)(═NR^{e4})NR^{c4}R^{d4};
or, R⁴ and L¹, together with the atoms to which they are attached, form a C₅₋₁₀ cycloalkyl, phenyl, 5-10 membered heterocycloalkyl, or 5-6 membered heteroaryl group;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c4} and R^{d4} attached to the same N atom, together with the N atom to which they are attached, form a 4-7 membered heterocycloalkyl group;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R⁵, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁-4 alkyl, (4-10 membered heterocycloalkyl)-C₁₋₄ alkyl, and (5-10 membered heteroaryl)-C₁₋₄ alkyl of R⁵ are each optionally substituted with 1, 2, 3, 4, 5, or 6 independently selected R^{5A} substituents;
each R^{5A} is independently selected from —S(O)R^{b5A}, —S(O)₂R^{b5A}, —S(O)NR^{c5A}R^{d5A}, and —S(O)₂NR^{c5A}R^{d5A};
each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁-4 alkyl, (4-10 membered heterocycloalkyl)-C₁₋₄ alkyl, and (5-10 membered heteroaryl)-C₁₋₄ alkyl;
or, any R^{c5} and R^{d5} attached to the same N atom, together with the N atom to which they are attached, form a 4-10 membered heterocycloalkyl group;
each R^{b5} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

In some embodiments:
X¹ is N, O, S, NR⁶, or CR⁷;
X² is N, O, S, NR⁸, or CR⁹;
X³ is C or N;
X⁴ is C or N;
Y is C or N;
Z is C or N;
each [symbol] is independently a single or double bond;
n is 0, 1, 2, 3, 4, 5, or 6;
m is 0, 1, 2, 3, 4, 5, or 6;
p is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is C₃₋₁₄ cycloalkyl, C₆₋₁₄ aryl, 4-14 membered heterocycloalkyl, or 5-14 membered heteroaryl;
L¹, L², and L³ are each independently selected from bond, C₁₋₆ alkylene, C₁₋₆ haloalkylene, C₃₋₇ cycloalkylene, 4-7 membered heterocycloalkylene, phenylene, 5-6 membered heteroarylene, —C₃₋₇ cycloalkylene-C₁₋₄ alkyl-, -(4-7 membered heterocycloalkylene)-C₁₋₄ alkyl-, -phenylene-C₁₋₄ alkyl-, -(5-6 membered heteroarylene)-C₁₋₄ alkyl-, —O—, —N(R^{L})—, —C(O)—, —N(R^{L})C(O)—, —N(R^{L})C(O)N(R^{L})—, —N(R^{L})C(O)O—, —S(O)—, —S(O)₂—, —S(O)(═NR^{L})—, —S(O)₂N(R^{L})—, and —N(R^{L})S(O)₂N(R^{L})—;
provided that when L³ is —C(O)—, —S(O)—, —S(O)₂—, or —S(O)(═NR^{L})—, then Ring C is C₉₋₁₄ cycloalkyl, C₁₀₋₁₄ aryl, 8-14 membered heterocycloalkyl, or 9-14 membered heteroaryl;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —NO₂, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1};
or, two R¹ together with the atoms to which they are attached form a C₃₋₇ cycloalkyl, phenyl, 4-7 membered heterocycloalkyl, or 5-6 membered heteroaryl group;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c1} and R^{d1} attached to the same N atom, together with the N atom to which they are attached, form a 4-10 membered heterocycloalkyl group;
each R^{b1} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2}NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —S(O)(═NR^{e2})R^{b2}, and —S(O)(═NR^{e2})R^{c2}R^{d2};
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c2} and R^{d2} attached to the same N atom, together with the N atom to which they are attached, form a 4-7 membered heterocycloalkyl group;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R³ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3}, —SR^{a3}, —NR^{c3}R^{d3}, —NO₂, —C(O)R^{a3}, —C(O)OR^{a3}, —C(O)NR^{c3}R^{d3}, —C(O)NR^{c3}(OR^{a3}), —OC(O)R^{a3}, —OC(O)NR^{c3}R^{d3}, —OC(O)OR^{a3}, —OS(O)₂R^{b3}, —OS(O)₂NR^{c3}R^{d3}, —NR^{c3}C(O)R^{a3}, —NR^{c3}C(O)OR^{a3}, —NR^{c3}C(O)NR^{c3}R^{d3}, —NR^{c3}S(O)₂R^{b3}, —NR^{c3}S(O)₂NR^{c3}R^{d3}, —NR^{c3}OR^{a3}, —NR^{c3}S(O)R^{b3}, —NR^{c3}S(O)NR^{c3}R^{d3}, —S(O)R^{b3}, —S(O)₂R^{b3}, —S(O)NR^{c3}R^{d3}, —S(O)₂NR^{c3}R^{d3}, —C(═NR^{e3})R^{a3}, —C(═NR^{e3})NR^{c3}R^{d3}, —NR^{c3}C(═NR^{e3})R^{a3}, —NR^{c3}C(═NR^{e3})NR^{c3}R^{d3}, —NR^{c3}S(O)(═NR^{e3})R^{b3}, —NR^{c3}S(O)(═NR^{e3})NR^{c3}R^{d3}, —OS(O)(═NR^{e3})R^{b3}, —S(O)(═NR^{e3})R^{b3}, —S(O)(═NR^{e3})NR^{c3}R^{d3}, —C(O)NR^{c3}S(O)₂R^{b3}, —C(O)NR^{c3}S(O)₂NR^{c3}R^{d3}, —S(O)₂NR^{c3}C(O)R^{b3}, —NR^{c3}S(O)NR^{c3}C(O)R^{b3}, and —P(O)R^{f3}R^{g3};
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c3} and R^{d3} attached to the same N atom, together with the N atom to which they are attached, form a 4-10 membered heterocycloalkyl group;
each R^{b3} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}, —NR^{c4}R^{d4}, —C(O)R^{a4}, —C(O)OR^{a4}, —C(O)NR^{c4}R^{d4}, —C(O)NR^{c4}(OR^{a4}), —OC(O)NR^{c4}R^{d4}, —NR^{c4}C(O)R^{a4}, —NR⁴C(O)OR^{a4}, —NR^{c4}C(O)NR^{c4}R^{d4}, —NR⁴S(O)₂R^{b4}, —NR^{c4}S(O)₂NR^{c4}R^{d4}, —NR^{c4}OR^{a4}, —NR^{c4}S(O)R^{b4}, —NR^{c4}S(O)NR^{c4}R^{d4}, —S(O)R^{b4}, —S(O)₂R¹⁴, —S(O)NR^{c4}R^{d4}, —S(O)₂NR^{c4}R^{d4}, —C(═NR^{e4})R^{a4}, —C(═NR^{e4})NR^{c4}R^{d4}, —NR^{c4}C(═NR^{e4})R^{a4}, —NR^{c4}C(═NR^{e4})NR^{c4}R^{d4}, —NR^{c4}S(O)(═NR^{e4})R^{b4}, —NR^{c4}S(O)(═NR^{e4})NR^{c4}R^{d4}, —S(O)(═NR^{e4})R^{b4}, and —S(O)(═NR^{e4})NR^{c4}R^{d4};
or, R⁴ and L¹, together with the atoms to which they are attached, form a C₅₋₁₀ cycloalkyl, phenyl, 5-10 membered heterocycloalkyl, or 5-6 membered heteroaryl group;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c4} and R^{d4} attached to the same N atom, together with the N atom to which they are attached, form a 4-7 membered heterocycloalkyl group;
each R^{c4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R⁵, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g6};
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c5} and R^{d5} attached to the same N atom, together with the N atom to which they are attached, form a 4-10 membered heterocycloalkyl group;
each R^{b5} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

In some embodiments:
X¹ is N, NR⁶, or CR⁷;
X² is N, NR⁸, or CR⁹;
X³ is C or N;
X⁴ is C or N;
Y is C or N;
Z is C or N;
each [symbol] is independently a single or double bond;
n 1, 2, or 3;
n is 0, 1, 2, or 3;
m is 0, 1, or 2;
p is 0, 1, 2, or 3;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is C₁₋₆ alkylene, —C(O)—, —S(O)—, —S(O)₂—, or —S(O)(═NR^{L});
provided that when L³ is —C(O)—, —S(O)—, —S(O)₂—, or —S(O)(═NR^{L})—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents;
each R^{5A} is independently selected from —S(O)R^{b5A}, —S(O)₂R^{b5A}, —S(O)NR^{c5A}R^{d5A}, —S(O)₂NR^{c5A}R^{d5A} and —P(O)R^{f5A}R^{g5A};
each R^{a5A}, R^{b5A}, R^{c5A}, R^{d5A}, R^{f5A}, and R^{g5A} is independently selected from H, C₁₋₆ alkyl, C₁₋₆ haloalkyl, C₂₋₆ alkenyl, C₂₋₆ alkynyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

In some embodiments:
X¹ is N, NR⁶, or CR⁷;
X² is N, NR⁸, or CR⁹;
X³ is C or N;
X⁴ is C or N;
Y is C or N;
Z is C or N;
each [symbol] is independently a single or double bond;
n is 0, 1, 2, or 3;
m is 0, 1, or 2;
p is 0, 1, 2, or 3;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is C₁₋₆ alkylene, —C(O)—, —S(O)—, —S(O)₂—, or —S(O)(═NR^{L});
provided that when L³ is —C(O)—, —S(O)—, —S(O)₂—, or —S(O)(═NR^{L})—, then Ring C is 8-14 membered heterocycloalkyl or 9-14 membered heteroaryl;
each R¹ is independently selected from halo, C₁₋₆ alkyl, and C₁₋₆ haloalkyl; each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents;
each R^{5A} is independently selected from —S(O)R^{b5A}, —S(O)₂R^{b5A}, —S(O)NR^{c5A}R^{d5A}, and —S(O)₂NR^{c5A}R^{d5A};
each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

In some embodiments:
X¹ is N, NR⁶, or CR⁷;
X² is N, NR⁸, or CR⁹;
X³ is C or N;
X⁴ is C or N;
Y is C or N;
Z is C or N;
each [symbol] is independently a single or double bond;
n is 0, 1, 2, or 3;
m is 0, 1, or 2;
p is 0, 1, 2, or 3;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is —C(O)—, —S(O)—, —S(O)₂—, or —S(O)(═NR^{L});
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

In some embodiments:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is C₁₋₆ alkylene, —C(O)—, —S(O)—, —S(O)₂—, or —S(O)(═NR^{L});
provided that when L³ is —C(O)—, —S(O)—, —S(O)₂—, or —S(O)(═NR^{L})—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents;
each R^{5A} is independently selected from —S(O)R^{b5A}, S(O)₂R^{b5A}, and —P(O)R^{f5A}R^{g5A}; and
each R^{b5A}, R^{f5} and R^{g5} is independently selected from H and C₁₋₆ alkyl.

In some embodiments:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is C₁₋₆ alkylene, —C(O)—, —S(O)—, —S(O)₂—, or —S(O)(═NR^{L});
provided that when L³ is —C(O)—, —S(O)—, —S(O)₂—, or —S(O)(═NR^{L})—, then Ring C is 8-14 membered heterocycloalkyl or 9-14 membered heteroaryl;
each R¹ is independently selected from halo, C₁₋₆ alkyl, and C₁₋₆ haloalkyl; each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents;
each R^{5A} is independently selected from —S(O)R^{b5A} and S(O)₂R^{b5A}; and
each R^{b5A} is independently selected from H and C₁₋₆ alkyl.

In some embodiments:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is —C(O)—, —S(O)—, —S(O)₂—, or —S(O)(═NR^{L});
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3}.
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl; and
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is —CH₂—;
L² is —NHC(O)—;
L³ is —CH₂— or —C(O)—;
provided that when L³ is —C(O)—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, and 5-10 membered heteroaryl, wherein the C₃₋₁₀ cycloalkyl, C₆₋₁₀ aryl, 4-10 membered heterocycloalkyl, and 5-10 membered heteroaryl of R⁵ are each optionally substituted with 1, 2, 3, or 4 independently selected R^{5A} substituents;
each R^{5A} is independently selected from —S(O)R^{b5A}, S(O)₂R^{b5A}, and —P(O)R^{f5A}R^{g5A}; and
each R^{b5A}, R^{f5A}, and R^{g5A} is independently selected from H and C₁₋₆ alkyl.

In some embodiments:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is —CH₂—;
L² is —NHC(O)—;
L³ is —CH₂— or —C(O)—;
provided that when L³ is —C(O)—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3}.
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, and 5-10 membered heteroaryl, wherein the C₃₋₁₀ cycloalkyl, C₆₋₁₀ aryl, 4-10 membered heterocycloalkyl, and 5-10 membered heteroaryl of R⁵ are each optionally substituted with 1, 2, 3, or 4 independently selected R^{5A} substituents;
each R^{5A} is independently selected from —S(O)R^{b5A} and S(O)₂R^{b5A}; and
each R^{b5A} is independently selected from H and C₁₋₆ alkyl.

In some embodiments:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl or 9-14 membered heteroaryl;
L¹ is —CH₂—;
L² is —NHC(O)—;
L³ is —C(O)—;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl; and
R⁵ is selected from C₃₋₁₀ cycloalkyl, C₆₋₁₀ aryl, 4-10 membered heterocycloalkyl, and 5-10 membered heteroaryl.

In some embodiments, the compound of Formula I is a compound of Formula II:

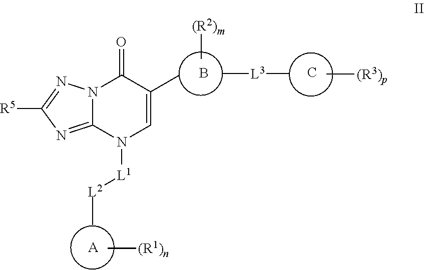

or a pharmaceutically acceptable salt thereof.

In some embodiments, the compound of Formula I is a compound of Formula III:

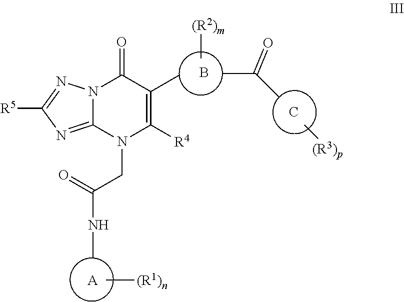

or a pharmaceutically acceptable salt thereof.

In some embodiments, the compound of Formula I is a compound of Formula

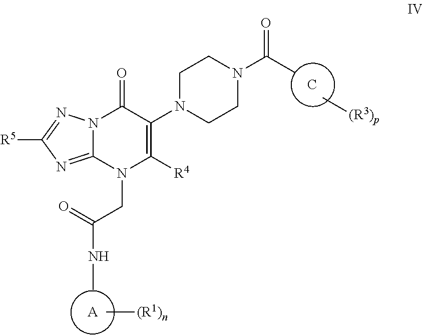

or a pharmaceutically acceptable salt thereof.

In some embodiments, the compound of Formula I is a compound of Formula V:

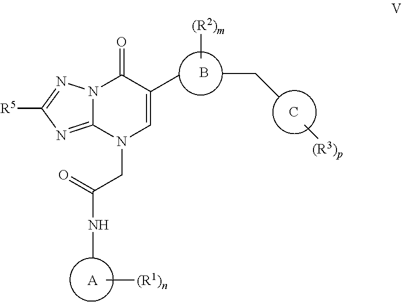

or a pharmaceutically acceptable salt thereof.

In some embodiments, the compound of Formula I is a compound of Formula VI:

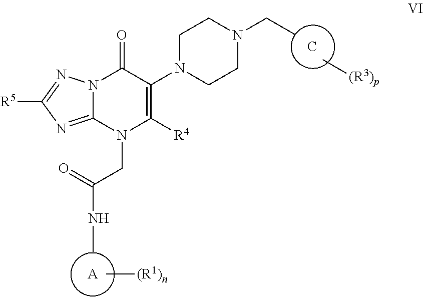

or a pharmaceutically acceptable salt thereof.

In some embodiments, the compound provided herein is selected from:
N-[2-chloro-4-(trifluoromethyl)phenyl]-2-[2-(3,6-dihydro-2H-pyran-4-yl)-5-ethyl-6-[4-(4-hydroxyisoquinoline-3-carbonyl)piperazin-1-yl]-7-oxo-[1,2,4]triazolo[1,5-a]pyrimidin-4-yl]acetamide;
N-[2-chloro-4-(trifluoromethyl)phenyl]-2-[2-(3,6-dihydro-2H-pyran-4-yl)-5-ethyl-6-[4-(3-hydroxyquinoline-2-carbonyl)piperazin-1-yl]-7-oxo-[1,2,4]triazolo[1,5-a]pyrimidin-4-yl]acetamide;
N-[2-chloro-4-(trifluoromethyl)phenyl]-2-[2-(3,6-dihydro-2H-pyran-4-yl)-5-ethyl-7-oxo-6-[4-(5H-pyrrolo[3,2-d]pyrimidine-4-carbonyl)piperazin-1-yl]-[1,2,4]triazolo[1,5-a]pyrimidin-4-yl]acetamide;
N-[2-chloro-4-(trifluoromethyl)phenyl]-2-[2-(3,6-dihydro-2H-pyran-4-yl)-5-ethyl-6-[4-[(3-hydroxy-2-pyridyl)methyl]piperazin-1-yl]-7-oxo-[1,2,4]triazolo[1,5-a]pyrimidin-4-yl]acetamide;
N-[2-chloro-4-(trifluoromethyl)phenyl]-2-[2-(3,6-dihydro-2H-pyran-4-yl)-5-ethyl-6-[4-(7-hydroxyquinoline-8-carbonyl)piperazin-1-yl]-7-oxo-[1,2,4]triazolo[1,5-a]pyrimidin-4-yl]acetamide;
N-(2-chloro-4-(trifluoromethyl)phenyl)-2-(5-ethyl-6-(4-(7-hydroxyquinoline-8-carbonyl)piperazin-1-yl)-7-oxo-2-(1-oxa-8-azaspiro[4.5]decan-8-yl)-[1,2,4]triazolo[1,5-a]pyrimidin-4(7H)-yl)acetamide;
N-(2-chloro-4-(trifluoromethyl)phenyl)-2-(5-ethyl-6-(4-((3-hydroxypyridin-2-yl)methyl)piperazin-1-yl)-7-oxo-2-(1-oxa-8-azaspiro[4.5]decan-8-yl)-[1,2,4]triazolo[1,5-a]pyrimidin-4(7H)-yl)acetamide;
N-(2-chloro-4-(trifluoromethyl)phenyl)-2-(5-ethyl-6-(4-(7-hydroxyquinoline-8-carbonyl)piperazin-1-yl)-2-(4-(methylsulfonyl)phenyl)-7-oxo-[1,2,4]triazolo[1,5-a]pyrimidin-4(7H)-yl)acetamide;
N-(2-chloro-4-(trifluoromethyl)phenyl)-2-(5-ethyl-6-(4-((3-hydroxypyridin-2-yl)methyl)piperazin-1-yl)-2-(4-(methylsulfonyl)phenyl)-7-oxo-[1,2,4]triazolo[1,5-a]pyrimidin-4(7H)-yl)acetamide; and
N-(2-chloro-4-(trifluoromethyl)phenyl)-2-(2-(4-(dimethylphosphoryl)phenyl)-5-ethyl-6-(4-((3-hydroxypyridin-2-yl)methyl)piperazin-1-yl)-7-oxo-[1,2,4]triazolo[1,5-a]pyrimidin-4(7H)-yl)acetamide

or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —N(R^{L})C(O)— includes both —N(R^{L})C(O)— and —C(O)N(R^{L})— (e.g. —NHC(O)— includes both —NHC(O)— and —C(O)NH—). Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ-n” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂.

In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-, 7-, 8-, 9-, or 10-membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-, 7-, 8-, 9-, or 10-membered monocyclic or bicyclic heteroaryl having 1, 2, 3, or 4 heteroatom ring members independently selected from N, O, and S. In some embodiments, the heteroaryl is a 5-6 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, and S. In some embodiments, the heteroaryl group contains 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group).

Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B).

The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, a “haloalkyl linking group” or “haloalkylene linking group” is a bivalent straight chain or branched haloalkyl linking group (“haloalkylene group”). Example haloalkylene groups include —CF₂—, —C₂F₄—, —CHF—, —CCl₂—, —CHCl—, —C₂Cl₄—, and the like.

As used herein, a “cycloalkyl linking group” or “cycloalkylene linking group” is a bivalent straight chain or branched cycloalkyl linking group (“cycloalkylene group”). Examples of “cycloalkyl linking groups” or “cycloalkylene groups” include cyclopropy-1,1,-diyl, cyclopropy-1,2-diyl, cyclobut-1,3,-diyl, cyclopent-1,3,-diyl, cyclopent-1,4,-diyl, cyclohex-1,2,-diyl, cyclohex-1,3,-diyl, cyclohex-1,4,-diyl, and the like.

As used herein, a “heterocycloalkyl linking group” or “heterocycloalkylene linking group” is a bivalent straight chain or branched heterocycloalkyl linking group (“heterocycloalkylene group”). Examples of “heterocycloalkyl linking groups” or “heterocycloalkylene groups” include azetidin-1,2-diyl, azetidin-1,3-diyl, pyrrolidin-1,2-diyl, pyrrolidin-1,3-diyl, pyrrolidin-2,3-diyl, piperidin-1,2-diyl, piperidin-1,3-diyl, piperidin-1,4-diyl, piperidin-2,3-diyl, piperidin-2,4-diyl, and the like.

As used herein, a “heteroaryl linking group” or “heteroarylene linking group” is a bivalent straight chain or branched heteroaryl linking group (“heteroarylene group”). Examples of “heteroaryl linking groups” or “heteroarylene groups” include pyrazol-1,3-diyl, imidazol-1,2,-diyl, pyridin-2,3-diyl, pyridin-2,4-diyl, pyridin-3,4-diyl, and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded.

For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{G}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid.

Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and according to various possible synthetic routes. Example synthetic methods for preparing compounds of the invention are provided in the Schemes below.

Compound of formula I-8 can be synthesized using a process shown in Scheme I. Condensation of compound I-1 and I-2 under suitable conditions can afford compound I-3. Alkylation of compound I-3 with appropriate halide I-4 under basic conditions can afford compound I-5. Compound I-6 can be access from compound I-5 via suitable coupling reactions (e.g., transition metal-catalyzed cross-coupling reactions). Deprotection of I-6 can give compound I-7. Functionalization of Ring B in compound I-7 via suitable reactions (e.g., amide coupling reaction) can give the product I-8.

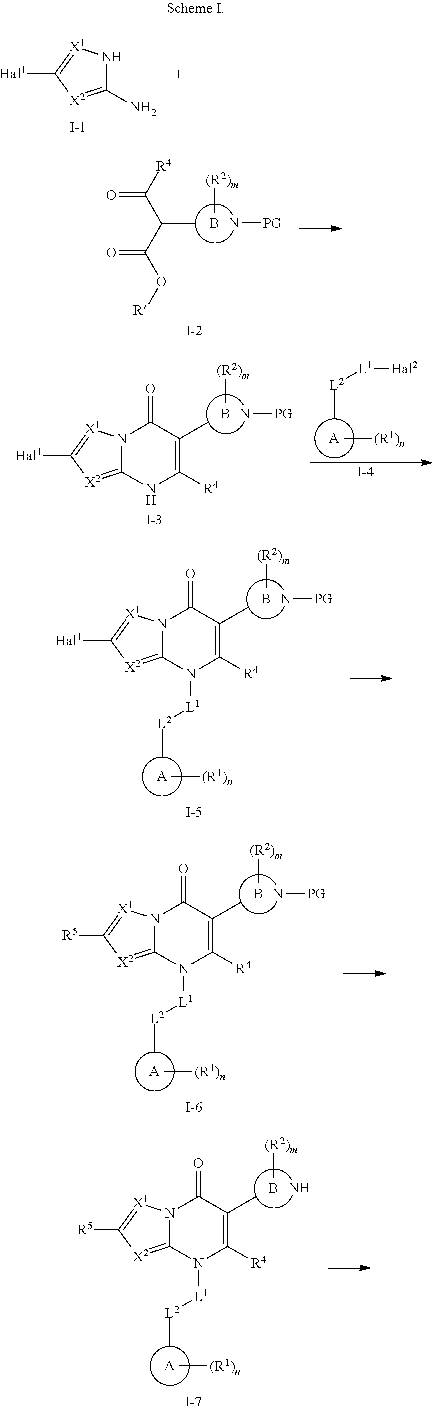

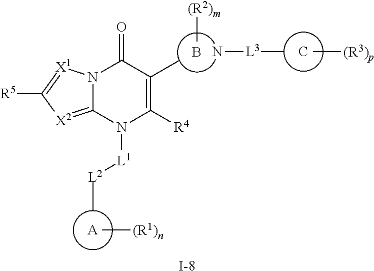

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

### Methods of Use

The present disclosure provides uses for compounds and compositions described herein. The compounds described herein can inhibit the activity of Werner syndrome helicase (WRN). In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by WRN. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with WRN. In some embodiments, the present disclosure provides methods of administering provided compounds or compositions to a subject suffering from or susceptible to a disease, disorder, or condition, wherein an underlying pathology is, wholly or partially, mediated by WRN.

In some embodiments, the compounds provided herein are useful as WRN inhibitors. In some embodiments, the present disclosure provides methods of inhibiting WRN in a subject comprising administering a provided compound or composition. In some embodiments, the present disclosure provides methods of inhibiting WRN in a biological sample comprising contacting the sample with a provided compound or composition.

In some embodiments, the present disclosure provides methods of treating a disease, disorder or condition associated with WRN in a subject in need thereof, comprising administering to the subject a compound, salt, or composition of the disclosure. In some embodiments, a disease, disorder or condition is associated with mutation of WRN. In some embodiments, the present disclosure provides methods of treating a disease, disorder or condition, wherein an underlying pathology is, wholly or partially, mediated by WRN, in a subject in need thereof, comprising administering to the subject a provided compound or composition.

In some embodiments, the present disclosure provides methods of treating a variety of WRN-dependent diseases and disorders. In some embodiments, the disease, disorder, or condition associated with WRN is a cancer.

In some embodiments, the disease, disorder, or condition associated with WRN is further associated with defective DNA mismatch repair (dMMR).

In some embodiments, the disease, disorder, or condition associated with WRN is further associated with microsatellite instability (MSI).

In some embodiments, the disease, disorder, or condition associated with WRN is further associated with microsatellite instability-high (MSI-H).

In some embodiments, the disease, disorder, or condition associated with WRN is further associated defective DNA mismatch repair (dMMR), microsatellite instability (MSI), or a combination thereof.

In some embodiments, the disease, disorder, or condition associated with WRN is further associated with defective DNA mismatch repair (dMMR) and microsatellite instability (MSI).

In some embodiments, the disease, disorder, or condition associated with WRN is further associated defective DNA mismatch repair (dMMR), microsatellite instability-high (MSI-H), or a combination thereof.

In some embodiments, the disease, disorder, or condition associated with WRN is further associated defective DNA mismatch repair (dMMR) and microsatellite instability-high (MSI-H).

In some embodiments, the disease, disorder, or condition is a cancer associated with defective DNA mismatch repair (dMMR). In some embodiments, the cancer is characterized, or has been characterized, as exhibiting defective DNA mismatch repair (dMMR).

In some embodiments, the disease, disorder, or condition is a cancer associated with microsatellite instability (MSI). In some embodiments, the cancer is characterized, or has been characterized, as exhibiting microsatellite instability (MSI).

In some embodiments, the disease, disorder, or condition is a cancer associated with microsatellite instability-high (MSI-H). In some embodiments, the cancer is characterized, or has been characterized, as exhibiting microsatellite instability-high (MSI-H).

In some embodiments, the disease, disorder, or condition is selected from colon cancer, small intestine cancer, endometrial cancer, gastric cancer, ovarian cancer, pancreatic cancer, cholangiocarcinoma, rectal cancer, adrenal cancer, breast cancer, uterine cancer, cervical cancer, Wilms tumor, mesothelioma, head and neck cancer, esophageal cancer, lung cancer, kidney cancer, sarcoma cancer, liver cancer, melanoma, prostate cancer, bladder cancer, glioblastoma, and neuroendocrine cancer.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” WRN with a compound described herein includes the administration of a compound described herein to an individual or patient, such as a human, having WRN, as well as, for example, introducing a compound described herein into a sample containing a cellular or purified preparation containing the WRN.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapy

One or more additional therapeutic agents such as, for example, chemotherapeutics or other anti-cancer agents useful for treating diseases associated with WRN can be used in combination with the compounds and salts provided herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: radiation therapies, DNA damage pathway inhibitors (including, but not limited to, PARP inhibitors, ATR inhibitors, DNAPK inhibitors, CHK1/2 inhibitors, and WEE1 inhibitors), immune checkpoint antibodies or inhibitors, or other immune activating therapies.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

In some embodiments, the additional therapeutic agent is administered simultaneously with a compound or salt provided herein. In some embodiments, the additional therapeutic agent is administered after administration of the compound or salt provided herein. In some embodiments, the additional therapeutic agent is administered prior to administration of the compound or salt provided herein. In some embodiments, the compound or salt provided herein is administered during a surgical procedure. In some embodiments, the compound or salt provided herein is administered in combination with an additional therapeutic agent during a surgical procedure.

As provided herein, the additional compounds, inhibitors, agents, etc. can be combined with the compounds provided herein in a single or continuous dosage form, or they can be administered simultaneously or sequentially as separate dosage forms.

### Pharmaceutical Formulations and Dosage Forms

When employed as pharmaceuticals, the compounds of the invention can be administered in the form of pharmaceutical compositions which refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

The liquid forms in which the compounds and compositions of the present invention can be incorporated for administration orally or by injection include aqueous solutions, suitably flavored syrups, aqueous or oil suspensions, and flavored emulsions with edible oils.

The compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are provided herein.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the WRN protein in tissue samples, including human, and for identifying WRN protein ligands by inhibition binding of a labeled compound. Accordingly, the present invention includes WRN biochemical assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³S, ³⁶C, ⁸²Br, ⁷⁵Br, ⁷⁶Br, ⁷⁷Br, 123I, ¹²⁴I, ¹²⁵I and ³¹I. The radionuclide that is incorporated in the instant radio-labeled compounds will depend on the specific application of that radio-labeled compound. For example, for in vitro WRN labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

One or more constituent atoms of the compounds presented herein can be replaced or substituted with isotopes of the atoms in natural or non-natural abundance. In some embodiments, one or more atoms are replaced or substituted by deuterium. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., the compound of any of Formulas I-IV) can be perdeuterated.

In some embodiments, the compound provided herein (e.g., the compound of any of Formulas I-IV), or a pharmaceutically acceptable salt thereof, comprises at least one deuterium atom.

In some embodiments, the compound provided herein (e.g., the compound of any of Formulas I-IV), or a pharmaceutically acceptable salt thereof, comprises two or more deuterium atoms.

In some embodiments, the compound provided herein (e.g., the compound of any of Formulas I-IV), or a pharmaceutically acceptable salt thereof, comprises three or more deuterium atoms.

In some embodiments, for a compound provided herein (e.g., the compound of any of Formulas I-IV), or a pharmaceutically acceptable salt thereof, all of the hydrogen atoms are replaced by deuterium atoms (i.e., the compound is “perdeuterated”).

It is understood that a “radio-labeled” or “labeled compound” is a compound that has incorporated at least one radionuclide. In some embodiments the radionuclide is selected from the group consisting of ³H, ¹⁴C, ¹²⁵I ³⁵S and ⁸²Br.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the WRN protein. Accordingly, the ability of a test compound to compete with the radio-labeled compound for binding to the WRN protein directly correlates to its binding affinity.

### Kits

The present invention also includes pharmaceutical kits useful, for example, in the treatment or prevention of WRN-associated diseases or disorders referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of WRN as described below.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity check.

Some of the compounds prepared were also separated on a preparative scale by reverse-phase high performance liquid chromatography (RP-HPLC) with MS detector or flash chromatography (silica gel) as indicated in the Examples.

The following abbreviations may be used herein: AcOH (acetic acid); Ac2O (acetic anhydride); aq. (aqueous); atm. (atmosphere(s)); Boc (t-butoxycarbonyl); BOP ((benzotriazol-1-yloxy)tris(dimethylamino)phosphonium hexafluorophosphate); br (broad); Cbz (carboxybenzyl); calc. (calculated); d (doublet); dd (doublet of doublets); DBU (1,8-diazabicyclo[5.4.0]undec-7-ene); DCM (dichloromethane); DIAD (N,N′-diisopropyl azidodicarboxylate); DIEA (N,N-diisopropylethylamine); DIPEA (N,N-diisopropylethylamine); DIBAL (diisobutylaluminium hydride); DMF (N,N-dimethylfornamide); Et (ethyl); EtOAc (ethyl acetate); EA (ethyl acetate); FCC (flash column chromatography); g (gram(s)); h (hour(s)); HATU (N,N,N′,N′-tetramethyl-O-(7-azabenzotriazol-1-yl)uronium hexafluorophosphate); HCl (hydrochloric acid); HPLC (high performance liquid chromatography); Hz (hertz); J (coupling constant); LCMS (liquid chromatography-mass spectrometry); LDA (lithium diisopropylamide); m (multiplet); M (molar); mCPBA (3-chloroperoxybenzoic acid); MS (Mass spectrometry); Me (methyl); MeCN (acetonitrile); MeOH (methanol); mg (milligram(s)); min. (minutes(s)); mL (milliliter(s)); mmol (millimole(s)); N (normal); NCS (N-chlorosuccinimide); NEt₃ (triethylamine); nM (nanomolar); NWP (N-methylpyrrolidinone); NMR (nuclear magnetic resonance spectroscopy); OTf (trifluoromethanesulfonate); Ph (phenyl); pM (picomolar); PPT (precipitate); RP-HPLC (reverse phase high performance liquid chromatography); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); pug (microgram(s)); μL (microliter(s)); μM (micromolar); wt % (weight percent). Brine is saturated aqueous sodium chloride. In vacuo is under vacuum.

### Example 1. N-[2-Chloro-4-(trifluoromethyl)phenyl]-2-[2-(3,6-dihydro-2H-pyran-4-yl)-5-ethyl-6-[4-(4-hydroxyisoquinoline-3-carbonyl)piperazin-1-yl]-7-oxo-[1,2,4]triazolo[1,5-a]pyrimidin-4-yl]acetamide

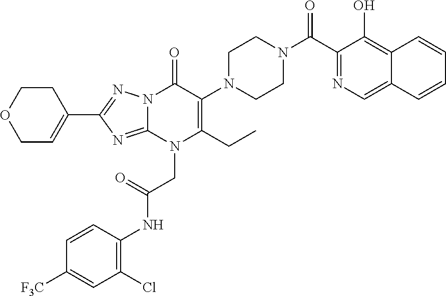 [SMILES: CCc1c(N2CCN(C(=O)c3ncc4ccccc4c3O)CC2)c(=O)n2nc(C3=CCOCC3)nc2n1CC(=O)Nc1ccc(C(F)(F)F)cc1Cl]

### Step 1: tert-butyl 4-(2-Bromo-5-ethyl-7-oxo-4H-[1,2,4]triazolo[1,5-a]pyrimidin-6-yl)piperazine-1-carboxylate

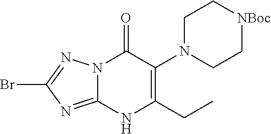 [SMILES: CCc1[nH]c2nc(Br)nn2c(=O)c1N1CCN(C(=O)OC(C)(C)C)CC1]

To a solution of 3-bromo-1H-1,2,4-triazol-5-amine (1.90 g, 9.55 mmol) in 20 ml EtOH was added tert-butyl 4-(1-ethoxycarbonyl-2-oxo-butyl)piperazine-1-carboxylate (2.51 g, 7.64 mmol), followed by phosphoric acid (1.03 g, 10.50 mmol). The resulting solution was heated to 80° C. and stirred overnight. Upon cooling to room temperature, the reaction mixture was concentrated and redissolved in 20 ml THF, then Boc₂O (2.29 g, 10.50 mmol) was added, followed by N-ethyl-N-isopropyl-propan-2-amine (5.00 mL, 28.64 mmol). The resulting solution was heated to 50° C. for 16 h. Upon cooling to room temperature, the resulting solution was diluted with 50 mL DCM and 50 mL water. The aqueous layer was extracted with DCM (3×50 mL). The combined organic layers were washed with brine (50 mL), and dried over anhydrous sodium sulfate. After filtration, the filtrate was concentrated under reduced pressure. The resulting residue was purified by silica gel column chromatography, eluting with ethyl acetate in DCM (0 to 100%), then MeOH in DCM (0 to 20%) to afford the desired product. LCMS calculated for C₁₆H₂₄BrN₆O₃(M+H)⁺ m/z=427.1; found 427.1.

### Step 2: tert-butyl 4-[2-Bromo-4-[2-[2-chloro-4-(trifluoromethyl)anilino]-2-oxo-ethyl]-5-ethyl-7-oxo-[1,2,4]triazolo[1,5-a]pyrimidin-6-yl]piperazine-1-carboxylate

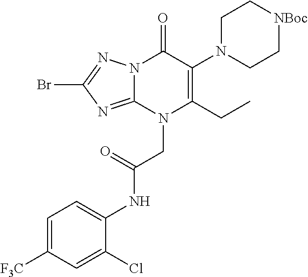 [SMILES: CCc1c(N2CCN(C(=O)OC(C)(C)C)CC2)c(=O)n2nc(Br)nc2n1CC(=O)Nc1ccc(C(F)(F)F)cc1Cl]

To a solution of tert-butyl 4-(2-bromo-5-ethyl-7-oxo-4H-[1,2,4]triazolo[1,5-a]pyrimidin-6-yl)piperazine-1-carboxylate (188.0 mg, 0.44 mmol) in 4 mL DMF was added 2-bromo-N-[2-chloro-4-(trifluoromethyl)phenyl]acetamide (167.1 mg, 0.53 mmol), followed by N-ethyl-N-isopropyl-propan-2-amine (0.24 mL, 1.32 mmol). The resulting solution was stirred at room temperature for 16 h, then poured into the mixture of 20 mL DCM and 20 mL water. The aqueous layer was extracted with DCM (3×20 mL). The combined organic layers were washed with brine (20 mL), and dried over anhydrous sodium sulfate. After filtration, the filtrate was concentrated under reduced pressure. The resulting residue was purified by silica gel column chromatography, eluting with ethyl acetate in DCM (0 to 100%) to afford the desired product. LCMS calculated for C₂₅H₂₈BrClF₃N₇O₄Na (M+Na)⁺ m/z=684.1 found 684.1.

### Step 3: tert-butyl 4-[4-[2-[2-Chloro-4-(trifluoromethyl)anilino]-2-oxo-ethyl]-2-(3,6-dihydro-2H-pyran-4-yl)-5-ethyl-7-oxo-[1,2,4]triazolo[1,5-a]pyrimidin-6-yl]piperazine-1-carboxylate

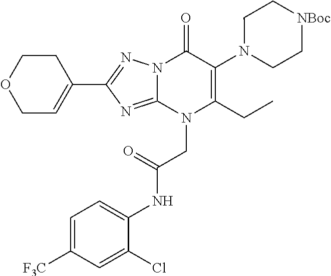 [SMILES: CCc1c(N2CCN(C(=O)OC(C)(C)C)CC2)c(=O)n2nc(C3=CCOCC3)nc2n1CC(=O)Nc1ccc(C(F)(F)F)cc1Cl]

To a solution of tert-butyl 4-[2-bromo-4-[2-[2-chloro-4-(trifluoromethyl)anilino]-2-oxo-ethyl]-5-ethyl-7-oxo-[1,2,4]triazolo[1,5-a]pyrimidin-6-yl]piperazine-1-carboxylate (103.0 mg, 0.16 mmol) in the mixture of 1.5 mL dioxane and 0.5 mL water was added [1,1′-bis(diphenylphosphino)ferrocene]dichloropalladium(II) (11.4 mg, 0.02 mmol), followed by cesium carbonate (151.9 mg, 0.47 mmol). The mixture was vacuumed and backfilled with nitrogen gas three times. The reaction mixture was then heated to 80° C. and 2-(3,6-dihydro-2H-pyran-4-yl)-4,4,5,5-tetramethyl-1,3,2-dioxaborolane (49 mg, 0.23 mmol) dissolved in 1 mL dioxane was added dropwise. The resulting mixture was stirred at 80° C. for 1 h, then poured into the mixture of 10 mL DCM and 10 mL water. The aqueous layer was extracted with DCM (3×10 mL). The combined organic layers were washed with brine (20 mL), and dried over anhydrous sodium sulfate. After filtration, the filtrate was concentrated under reduced pressure. The resulting residue was purified by silica gel column chromatography, eluted with ethyl acetate in DCM (0 to 100%) to afford the desired product. LCMS calculated for C₃₀H₃₆ClF₃N₇O₅ (M+H)⁺ m/z=666.2 found 666.2.

### Step 4: N-[2-Chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide

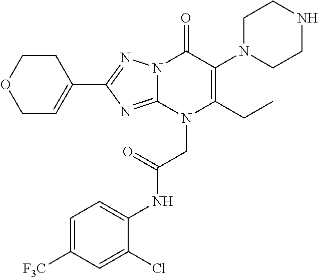 [SMILES: CCc1c(N2CCNCC2)c(=O)n2nc(C3=CCOCC3)nc2n1CC(=O)Nc1ccc(C(F)(F)F)cc1Cl]

To a solution of tert-butyl 4-[4-[2-[2-chloro-4-(trifluoromethyl)anilino]-2-oxo-ethyl]-2-(3,6-dihydro-2H-pyran-4-yl)-5-ethyl-7-oxo-[1,2,4]triazolo[1,5-a]pyrimidin-6-yl]piperazine-1-carboxylate (85 mg, 0.13 mmol) in 2 mL DCM was added 0.5 ml TFA dropwise. The resulting solution was stirred at r.t. for 30 min, then directly concentrated to give the crude product, which was directly used without further purification. LCMS calculated for C₂₅H₂₈ClF₃N₇O₃ (M+H)⁺ m/z=566.2 found 566.2.

### Step 5: N-[2-Chloro-4-(trifluoromethyl)phenyl]-2-[2-(3,6-dihydro-2H-pyran-4-yl)-5-ethyl-6-[4-(4-hydroxyisoquinoline-3-carbonyl)piperazin-1-yl]-7-oxo-[1,2,4]triazolo[1,5-a]pyrimidin-4-yl]acetamide

To a solution of N-[2-chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide (10.0 mg, 0.02 mmol) in 0.5 mL DMF was added 4-hydroxyisoquinoline-3-carboxylic acid (6.7 mg, 0.04 mmol) and N-ethyl-N-isopropyl-propan-2-amine (0.02 mL, 0.09 mmol), followed by HATU (10.1 mg, 0.03 mmol). The resulting solution was stirred at r.t. for 2 h. The mixture was then diluted with acetonitrile and water, which was then purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₅H₃₃ClF₃N₈O₅(M+H)⁺ m/z=737.2; found 737.3.

### Example 2. N-[2-Chloro-4-(trifluoromethyl)phenyl]-2-[2-(3,6-dihydro-2H-pyran-4-yl)-5-ethyl-6-[4-(3-hydroxyquinoline-2-carbonyl)piperazin-1-yl]-7-oxo-[1,2,4]triazolo[1,5-a]pyrimidin-4-yl]acetamide

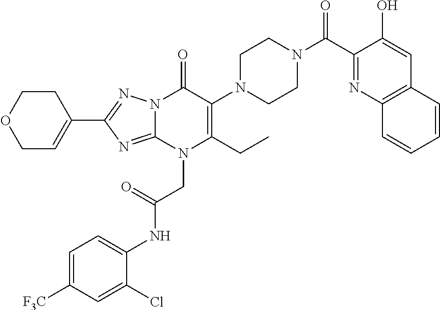 [SMILES: CCc1c(N2CCN(C(=O)c3nc4ccccc4cc3O)CC2)c(=O)n2nc(C3=CCOCC3)nc2n1CC(=O)Nc1ccc(C(F)(F)F)cc1Cl]

The title compound was prepared using similar procedures as described for Example 1 with 3-hydroxyquinoline-2-carboxylic acid replacing 4-hydroxyisoquinoline-3-carboxylic acid in Step 5. LCMS calculated for C₃₅H₃₃ClF₃N₈O₅ (M+H)⁺ m/z=737.2; found 737.3.

### Example 3: N-[2-chloro-4-(trifluoromethyl)phenyl]-2-[2-(3,6-dihydro-2H-pyran-4-yl)-5-ethyl-7-oxo-6-[4-(5H-pyrrolo[3,2-d]pyrimidine-4-carbonyl)piperazin-1-yl]-[1,2,4]triazolo[1,5-a]pyrimidin-4-yl]acetamide

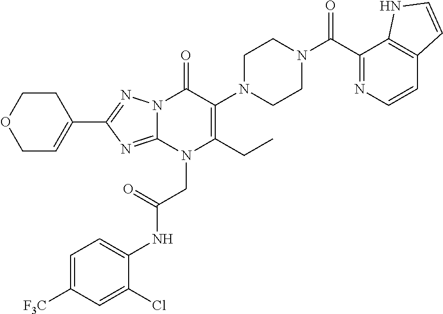 [SMILES: CCc1c(N2CCN(C(=O)c3nccc4cc[nH]c34)CC2)c(=O)n2nc(C3=CCOCC3)nc2n1CC(=O)Nc1ccc(C(F)(F)F)cc1Cl]

The titled compound was prepared using similar procedures as described for Example 1 with 5H-pyrrolo[3,2-d]pyrimidine-4-carboxylic acid replacing 4-hydroxyisoquinoline-3-carboxylic acid in Step 5. LCMS calculated for C₃₂H₃₁ClF₃N₁₀O₄(M+H)⁺ m/z=711.2; found 711.2.

### Example 4. N-[2-chloro-4-(trifluoromethyl)phenyl]-2-[2-(3,6-dihydro-2H-pyran-4-yl)-5-ethyl-6-[4-[(3-hydroxy-2-pyridyl)methyl]piperazin-1-yl]-7-oxo-[1,2,4]triazolo[1,5-a]pyrimidin-4-yl]acetamide

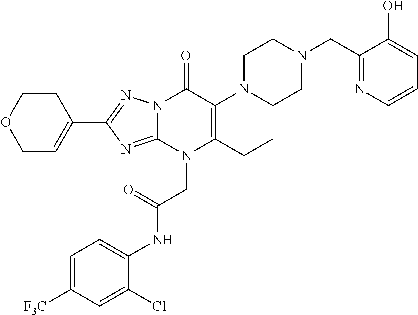 [SMILES: CCc1c(N2CCN(Cc3ncccc3O)CC2)c(=O)n2nc(C3=CCOCC3)nc2n1CC(=O)Nc1ccc(C(F)(F)F)cc1Cl]

To a solution of N-[2-chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide (10.0 mg, 0.02 mmol) in 1 mL DMF was added 2-(bromomethyl)pyridin-3-ol hydrobromide salt (5.7 mg, 0.02 mmol), followed by N-ethyl-N-isopropyl-propan-2-amine (0.02 mL, 0.14 mmol). The resulting solution was stirred at r.t. for 1 hour. The mixture was then diluted with acetonitrile and water, then purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide a TFA salt of the desired product as a white solid. LCMS calculated for C₃₁H₃₃ClF₃N₈O₄(M+H)⁺ m/z=673.2; found 673.2.

### Example 5. N-[2-chloro-4-(trifluoromethyl)phenyl]-2-[2-(3,6-dihydro-2H-pyran-4-yl)-5-ethyl-6-[4-(7-hydroxyquinoline-8-carbonyl)piperazin-1-yl]-7-oxo-[1,2,4]triazolo[1,5-a]pyrimidin-4-yl]acetamide

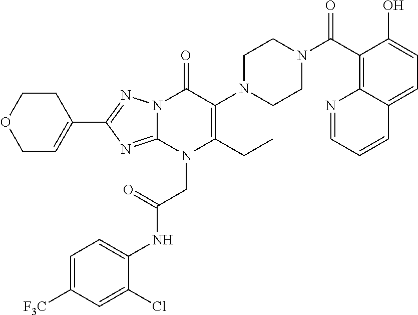 [SMILES: CCc1c(N2CCN(C(=O)c3c(O)ccc4cccnc34)CC2)c(=O)n2nc(C3=CCOCC3)nc2n1CC(=O)Nc1ccc(C(F)(F)F)cc1Cl]

The title compound was prepared using a similar procedure as described for Example 1, with 7-hydroxyquinoline-8-carboxylic acid replacing 4-hydroxyisoquinoline-3-carboxylic acid in Step 5. LCMS calculated for C₃₅H₃₃ClF₃N₈O₅ (M+H)⁺ m/z=737.2; found 737.3.

### Example 6. N-(2-chloro-4-(trifluoromethyl)phenyl)-2-(5-ethyl-6-(4-(7-hydroxyquinoline-8-carbonyl)piperazin-1-yl)-7-oxo-2-(1-oxa-8-azaspiro[4.5]decan-8-yl)-[1,2,4]triazolo[1,5-a]pyrimidin-4(7H)-yl)acetamide

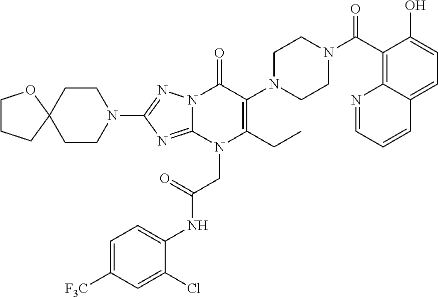 [SMILES: CCc1c(N2CCN(C(=O)c3c(O)ccc4cccnc34)CC2)c(=O)n2nc(N3CCC4(CCCO4)CC3)nc2n1CC(=O)Nc1ccc(C(F)(F)F)cc1Cl]

### Step 1: tert-butyl 4-(4-(2-((2-chloro-4-(trifluoromethyl)phenyl)amino)-2-oxoethyl)-5-ethyl-7-oxo-2-(1-oxa-8-azaspiro[4.5]decan-8-yl)-4,7-dihydro-[1,2,4]triazolo[1,5-a]pyrimidin-6-yl)piperazine-1-carboxylate

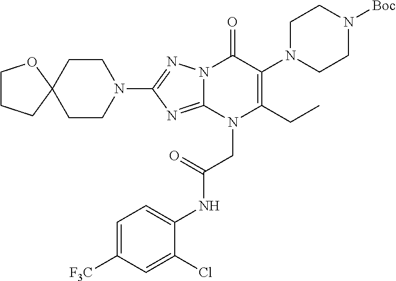 [SMILES: CCc1c(N2CCN(C(=O)OC(C)(C)C)CC2)c(=O)n2nc(N3CCC4(CCCO4)CC3)nc2n1CC(=O)Nc1ccc(C(F)(F)F)cc1Cl]

To a solution of tert-butyl 4-[2-bromo-4-[2-[2-chloro-4-(trifluoromethyl)anilino]-2-oxo-ethyl]-5-ethyl-7-oxo-[1,2,4]triazolo[1,5-a]pyrimidin-6-yl]piperazine-1-carboxylate (70 mg, 0.11 mmol) in DMSO (0.5 mL) was added 1-oxa-8-azaspiro[4.5]decane hydrochloride (75 mg, 0.42 mmol) and potassium acetate (104 mg, 1.06 mmol). The mixture was stirred at 120° C. for 4 hours, then cooled to room temperature. The resulting solution was diluted with water and ethyl acetate.

The aqueous layer was extracted with EtOAc. The combined organic layers were dried over anhydrous magnesium sulfate, filtered, then concentrated under reduced pressure. The resulting residue was purified by silica gel column chromatography, eluting with 0 to 100% EtOAc in hexanes to afford the desired product. LCMS calculated for C₃₃H₄₃ClF₃N₈O₅ (M+H)⁺ m/z=723.3; found 723.3.

### Step 2: N-(2-chloro-4-(trifluoromethyl)phenyl)-2-(5-ethyl-7-oxo-6-(piperazin-1-yl)-2-(1-oxa-8-azaspiro[4.5]decan-8-yl)-[1,2,4]triazolo[1,5-a]pyrimidin-4(7H)-yl)acetamide

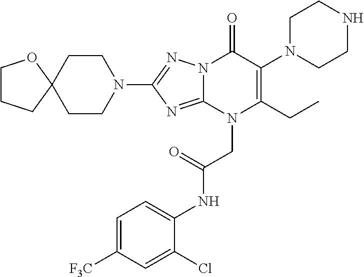 [SMILES: CCc1c(N2CCNCC2)c(=O)n2nc(N3CCC4(CCCO4)CC3)nc2n1CC(=O)Nc1ccc(C(F)(F)F)cc1Cl]

The title compound was prepared using a similar procedure as described for Example 1, Step 4, with tert-butyl 4-(4-(2-((2-chloro-4-(trifluoromethyl)phenyl)amino)-2-oxoethyl)-5-ethyl-7-oxo-2-(1-oxa-8-azaspiro[4.5]decan-8-yl)-4,7-dihydro-[1,2,4]triazolo[1,5-a]pyrimidin-6-yl)piperazine-1-carboxylate replacing tert-butyl 4-[4-[2-[2-chloro-4-(trifluoromethyl)anilino]-2-oxo-ethyl]-2-(3,6-dihydro-2H-pyran-4-yl)-5-ethyl-7-oxo-[1,2,4]triazolo[1,5-a]pyrimidin-6-yl]piperazine-1-carboxylate. LCMS calculated for C₂₈H₃₅ClF₃N₈O₃ (M+H)⁺ m/z=623.3; found 623.2.

### Step 3: N-(2-chloro-4-(trifluoromethyl)phenyl)-2-(5-ethyl-6-(4-(7-hydroxyquinoline-8-carbonyl)piperazin-1-yl)-7-oxo-2-(1-oxa-8-azaspiro[4.5]decan-8-yl)-[1,2,4]triazolo[1,5-a]pyrimidin-4(7H)-yl)acetamide

To a solution of N-(2-chloro-4-(trifluoromethyl)phenyl)-2-(5-ethyl-7-oxo-6-(piperazin-1-yl)-2-(1-oxa-8-azaspiro[4.5]decan-8-yl)-[1,2,4]triazolo[1,5-a]pyrimidin-4(7H)-yl)acetamide (13.8 mg, 0.02 mmol) in NMP (0.5 mL) was added 7-hydroxyquinoline-8-carboxylic acid (5.44 mg, 0.03 mmol) and HATU (12.6 mg, 0.03 mmol). To this mixture was added DIPEA (20 μL, 0.11 mmol) dropwise. The resulting solution was stirred at room temperature for 15 minutes, then dimethylamine (2.0 M in THF, 50 μL) was added. The mixture was stirred at room temperature for 15 minutes, then diluted with acetonitrile and purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₈H₄₀ClF₃N₉O₅ (M+H)⁺ m/z=794.3; found 794.3.

### Example 7: N-(2-chloro-4-(trifluoromethyl)phenyl)-2-(5-ethyl-6-(4-((3-hydroxypyridin-2-yl)methyl)piperazin-1-yl)-7-oxo-2-(1-oxa-8-azaspiro[4.5]decan-8-yl)-[1,2,4]triazolo[1,5-a]pyrimidin-4(7H)-yl)acetamide

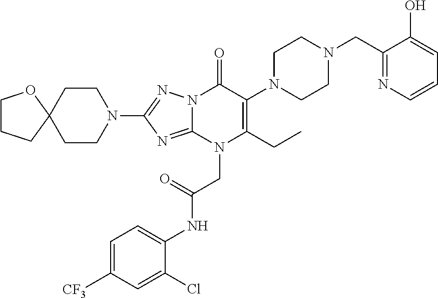 [SMILES: CCc1c(N2CCN(Cc3ncccc3O)CC2)c(=O)n2nc(N3CCC4(CCCO4)CC3)nc2n1CC(=O)Nc1ccc(C(F)(F)F)cc1Cl]

The title compound was prepared using a similar procedure as described for Example 4, with N-(2-chloro-4-(trifluoromethyl)phenyl)-2-(5-ethyl-7-oxo-6-(piperazin-1-yl)-2-(1-oxa-8-azaspiro[4.5]decan-8-yl)-[1,2,4]triazolo[1,5-a]pyrimidin-4(7H)-yl)acetamide (as prepared in Example 6) replacing N-[2-chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide. LCMS calculated for C₃₄H₄₀ClF₃N₉O₄ (M+H)⁺ m/z=730.3; found 730.3.

### Example 8. N-(2-Chloro-4-(trifluoromethyl)phenyl)-2-(5-ethyl-6-(4-(7-hydroxyquinoline-8-carbonyl)piperazin-1-yl)-2-(4-(methylsulfonyl)phenyl)-7-oxo-[1,2,4]triazolo[1,5-a]pyrimidin-4(7H)-yl)acetamide

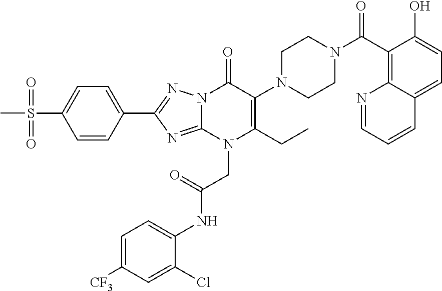 [SMILES: CCc1c(N2CCN(C(=O)c3c(O)ccc4cccnc34)CC2)c(=O)n2nc(-c3ccc(S(C)(=O)=O)cc3)nc2n1CC(=O)Nc1ccc(C(F)(F)F)cc1Cl]

### Step 1: tert-butyl 4-(4-(2-((2-chloro-4-(trifluoromethyl)phenyl)amino)-2-oxoethyl)-5-ethyl-2-(4-(methylsulfonyl)phenyl)-7-oxo-4,7-dihydro-[1,2,4]triazolo[1,5-a]pyrimidin-6-yl)piperazine-1-carboxylate

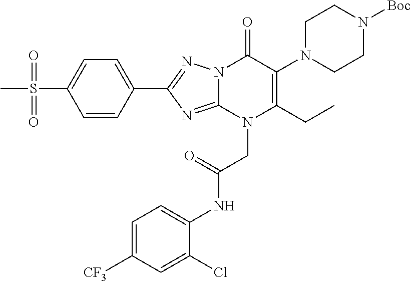 [SMILES: CCc1c(N2CCN(C(=O)OC(C)(C)C)CC2)c(=O)n2nc(-c3ccc(S(C)(=O)=O)cc3)nc2n1CC(=O)Nc1ccc(C(F)(F)F)cc1Cl]

A solution of tert-butyl 4-[2-bromo-4-[2-[2-chloro-4-(trifluoromethyl)anilino]-2-oxo-ethyl]-5-ethyl-7-oxo-[1,2,4]triazolo[1,5-a]pyrimidin-6-yl]piperazine-1-carboxylate (30.0 mg, 45 μmol), (4-methylsulfonylphenyl)boronic acid (11.8 mg, 59 μmol), sodium carbonate (9.6 mg, 91 μmol), and [1,1′-bis(diphenylphosphino)ferrocene]dichloropalladium(II) (7.4 mg, 9.1 μmol) in a mixture of 1.5 mL dioxane and 0.3 mL water was evacuated and backfilled with nitrogen gas three times. The reaction mixture was heated at 80° C. for 1 h, then cooled to room temperature. The resulting solution was diluted with water and ethyl acetate. The aqueous layer was extracted with EtOAc. The combined organic layers were dried over anhydrous magnesium sulfate, filtered, then concentrated under reduced pressure. The resulting residue was purified by silica gel column chromatography, eluting with 0 to 100% EtOAc in hexanes to afford the desired product. LCMS calculated for C₃₂H₃₆ClF₃N₇O₆S (M+H)⁺ m/z=738.2; found (M-56+H)+682.1.

### Step 2: N-(2-chloro-4-(trifluoromethyl)phenyl)-2-(5-ethyl-2-(4-(methylsulfonyl)phenyl)-7-oxo-6-(piperazin-1-yl)-[1,2,4]triazolo[1,5-a]pyrimidin-4(7H)-yl)acetamide

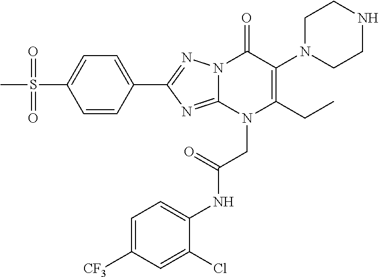 [SMILES: CCc1c(N2CCNCC2)c(=O)n2nc(-c3ccc(S(C)(=O)=O)cc3)nc2n1CC(=O)Nc1ccc(C(F)(F)F)cc1Cl]

The title compound was prepared using a similar procedure as described for Example 1, Step 4, with tert-butyl 4-(4-(2-((2-chloro-4-(trifluoromethyl)phenyl)amino)-2-oxoethyl)-5-ethyl-2-(4-(methylsulfonyl)phenyl)-7-oxo-4,7-dihydro-[1,2,4]triazolo[1,5-a]pyrimidin-6-yl)piperazine-1-carboxylate replacing tert-butyl 4-[4-[2-[2-chloro-4-(trifluoromethyl)anilino]-2-oxo-ethyl]-2-(3,6-dihydro-2H-pyran-4-yl)-5-ethyl-7-oxo-[1,2,4]triazolo[1,5-a]pyrimidin-6-yl]piperazine-1-carboxylate. LCMS calculated for C₂₇H₂₈ClF₃N₇O₄S (M+H)⁺ m/z=638.2; found 638.1.

### Step 3: N-(2-chloro-4-(trifluoromethyl)phenyl)-2-(5-ethyl-6-(4-(7-hydroxyquinoline-8-carbonyl)piperazin-1-yl)-2-(4-(methylsulfonyl)phenyl)-7-oxo-[1,2,4]triazolo[1,5-a]pyrimidin-4(7H)-yl)acetamide

The title compound was prepared using a similar procedure as described for Example 5, with N-(2-chloro-4-(trifluoromethyl)phenyl)-2-(5-ethyl-2-(4-(methylsulfonyl)phenyl)-7-oxo-6-(piperazin-1-yl)-[1,2,4]triazolo[1,5-a]pyrimidin-4(7H)-yl)acetamide replacing N-[2-Chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide. LCMS calculated for C₃₇H₃₃ClF₃N₈O₆S (M+H)⁺ m/z=809.2; found 809.1.

### Example 9. N-(2-Chloro-4-(trifluoromethyl)phenyl)-2-(5-ethyl-6-(4-((3-hydroxypyridin-2-yl)methyl)piperazin-1-yl)-2-(4-(methylsulfonyl)phenyl)-7-oxo-[1,2,4]triazolo[1,5-a]pyrimidin-4(7H)-yl)acetamide

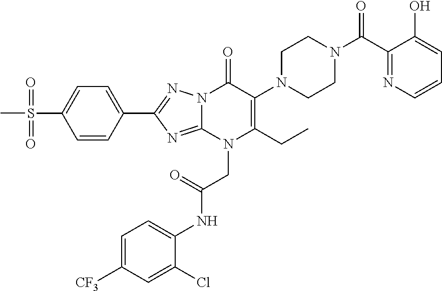 [SMILES: CCc1c(N2CCN(C(=O)c3ncccc3O)CC2)c(=O)n2nc(-c3ccc(S(C)(=O)=O)cc3)nc2n1CC(=O)Nc1ccc(C(F)(F)F)cc1Cl]

The title compound was prepared using a similar procedure as described for Example 4, with N-(2-chloro-4-(trifluoromethyl)phenyl)-2-(5-ethyl-2-(4-(methylsulfonyl)phenyl)-7-oxo-6-(piperazin-1-yl)-[1,2,4]triazolo[1,5-a]pyrimidin-4(7H)-yl)acetamide (Example 8, Step 2) replacing N-[2-chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide. LCMS calculated for C₃₃H₃₃ClF₃N₈O₅S (M+H)⁺ m/z=745.2; found 745.2.

### Example 10. N-(2-Chloro-4-(trifluoromethyl)phenyl)-2-(2-(4-(dimethylphosphoryl)phenyl)-5-ethyl-6-(4-((3-hydroxypyridin-2-yl)methyl)piperazin-1-yl)-7-oxo-[1,2,4]triazolo[1,5-a]pyrimidin-4(7H)-yl)acetamide

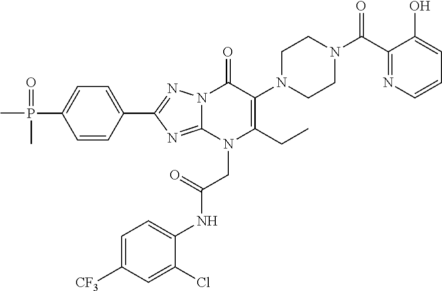 [SMILES: CCc1c(N2CCN(C(=O)c3ncccc3O)CC2)c(=O)n2nc(-c3ccc(P(C)(C)=O)cc3)nc2n1CC(=O)Nc1ccc(C(F)(F)F)cc1Cl]

### Step 1: tert-butyl 4-(4-(2-((2-chloro-4-(trifluoromethyl)phenyl)amino)-2-oxoethyl)-2-(4-(dimethylphosphoryl)phenyl)-5-ethyl-7-oxo-4,7-dihydro-[1,2,4]triazolo[1,5-a]pyrimidin-6-yl)piperazine-1-carboxylate

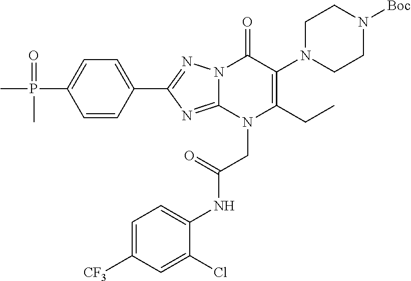 [SMILES: CCc1c(N2CCN(C(=O)OC(C)(C)C)CC2)c(=O)n2nc(-c3ccc(P(C)(C)=O)cc3)nc2n1CC(=O)Nc1ccc(C(F)(F)F)cc1Cl]

The title compound was prepared using a similar procedure as described for Example 8, Step 1, with dimethyl(4-(4,4,5,5-tetramethyl-1,3,2-dioxaborolan-2-yl)phenyl)phosphine oxide replacing (4-methylsulfonylphenyl)boronic acid. LCMS calculated for C₃₃H₃₉ClF₃N₇O₅P (M+H)⁺ m/z=736.2; found 736.2.

### Step 2: N-(2-chloro-4-(trifluoromethyl)phenyl)-2-(2-(4-(dimethylphosphoryl)phenyl)-5-ethyl-7-oxo-6-(piperazin-1-yl)-[1,2,4]triazolo[1,5-a]pyrimidin-4(7H)-yl)acetamide

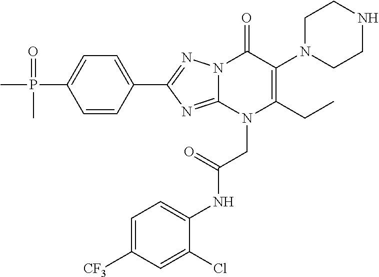 [SMILES: CCc1c(N2CCNCC2)c(=O)n2nc(-c3ccc(P(C)(C)=O)cc3)nc2n1CC(=O)Nc1ccc(C(F)(F)F)cc1Cl]

The title compound was prepared using a similar procedure as described for Example 1, Step 4, with tert-butyl 4-(4-(2-((2-chloro-4-(trifluoromethyl)phenyl)amino)-2-oxoethyl)-2-(4-(dimethylphosphoryl)phenyl)-5-ethyl-7-oxo-4,7-dihydro-[1,2,4]triazolo[1,5-a]pyrimidin-6-yl)piperazine-1-carboxylate replacing tert-butyl 4-[4-[2-[2-chloro-4-(trifluoromethyl)anilino]-2-oxo-ethyl]-2-(3,6-dihydro-2H-pyran-4-yl)-5-ethyl-7-oxo-[1,2,4]triazolo[1,5-a]pyrimidin-6-yl]piperazine-1-carboxylate. LCMS calculated for C₂₈H₃₁ClF₃N₇O₃P (M+H)⁺ m/z=636.2; found 636.2.

### Step 3: N-(2-chloro-4-(trifluoromethyl)phenyl)-2-(2-(4-(dimethylphosphoryl)phenyl)-5-ethyl-6-(4-((3-hydroxypyridin-2-yl)methyl)piperazin-1-yl)-7-oxo-[1,2,4]triazolo[1,5-a]pyrimidin-4(7H)-yl)acetamide

The title compound was prepared using a similar procedure as described for Example 4, with N-(2-chloro-4-(trifluoromethyl)phenyl)-2-(2-(4-(dimethylphosphoryl)phenyl)-5-ethyl-7-oxo-6-(piperazin-1-yl)-[1,2,4]triazolo[1,5-a]pyrimidin-4(7H)-yl)acetamide replacing N-[2-chloro-4-(trifluoromethyl)phenyl]-2-[2-(3,6-dihydro-2H-pyran-4-yl)-5-ethyl-7-oxo-6-piperazin-1-yl-[1,2,4]triazolo[1,5-a]pyrimidin-4-yl]acetamide. LCMS calculated for C₃₄H₃₆ClF₃N₈O₄P (M+H)⁺ m/z=743.2; found 743.2.

### Example A. Cell-Titer Glo Measurement of Growth-Inhibitory Activity in SW48 Cells

This cell-based assay measures growth-inhibitory activity of selected WRN inhibitors in SW48 colorectal cancer cells. SW48 cell line (#CCL-231) was purchased from American type culture collection (ATCC). Cells were cultured following ATCC instructions. The base medium for this cell line is ATCC-formulated Leibovitz's L-15 Medium (#30-2008). To prepare complete medium, fetal bovine serum was added at a final concentration of 10%. Antibiotics including penicillin and streptomycin (#SV30010) were purchased from Cytiva and added into complete medium to prevent bacteria contamination. Cells with a passage number of below 20 were used in this cell-based assay. One day before the assay, cells were resuspended in complete medium and seeded into 96-well plates at a final density of 2000 cells per well. One extra plate was added to measure baseline viability on day 1 before compound treatment. Baseline cell viability was measured using Promega Cell-titer Glo reagent (#G7573) following the protocol provided by the manufacture. Compounds were dissolved in DMSO at a stock concentration of 10 mmol/L. Three-fold serial dilution was performed using DMSO to prepare master plates. Compounds were further diluted using complete medium and added into assay plates. The final concentration of DMSO was 0.2% in assay plates. Cell viability was measured on day 5 using Promega Cell-titer Glo reagent following the same protocol provided by the manufacture. Assay plates were kept on orbital shaker (300 rounds per min) for 2 min and then kept in 37° C. incubator for another 10 minutes. Cell-titer Glo luminescence signal was measured using i3x plate reader. Baseline cell viability was subtracted for growth inhibition 50 (GI₅₀) estimate using GraphPad Prism following the four-parameter logistic (4PL) curve-fitting mathematical model.

Results of the assay described above are presented in Table 1. “+” indicates an GI₅₀ less than 100 nM; “++” indicates an GI₅₀ greater than or equal to 100 nM but less than 1000 nM; “+++” indicates an GI₅₀ greater than or equal to 1000 nM but less than 5000 nM; and “++++” indicates an GI₅₀ greater than or equal to 5000 nM.

TABLE 1

| Example | SW48_GI₅₀ (nM) |
|---|---|
| 1 | + |
| 2 | + |
| 3 | ++ |
| 4 | ++ |
| 5 | + |
| 6 | ++ |
| 7 | +++ |
| 8 | + |
| 9 | ++ |
| 10 | + |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 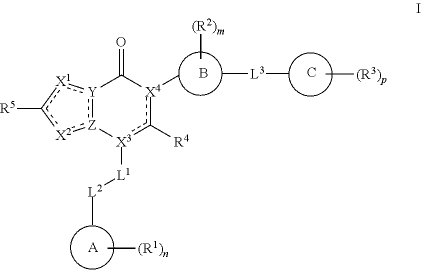
or a pharmaceutically acceptable salt thereof, wherein:
X¹ is N, O, S, NR⁶, or CR⁷;
X² is N, O, S, NR⁸, or CR⁹;
X³ is C or N;
X⁴ is C or N;
Y is C or N;
Z is C or N;
each [symbol] is independently a single or double bond;
n is 0, 1, 2, 3, 4, 5, or 6;
m is 0, 1, 2, 3, 4, 5, or 6;
p is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is C₃₋₁₄ cycloalkyl, C₆₋₁₄ aryl, 4-14 membered heterocycloalkyl, or 5-14 membered heteroaryl;
L¹, L², and L³ are each independently selected from bond, C₁₋₆ alkylene, C₁₋₆ haloalkylene, C₃₋₇ cycloalkylene, 4-7 membered heterocycloalkylene, phenylene, 5-6 membered heteroarylene, —C₃₋₇ cycloalkylene-C₁₋₄ alkyl-, -(4-7 membered heterocycloalkylene)-C₁₋₄ alkyl-, -phenylene-C₁₋₄ alkyl-, -(5-6 membered heteroarylene)-C₁₋₄ alkyl-, —O—, —N(R^{L})—, —C(O)—, —N(R^{L})C(O)—, —N(R^{L})C(O)N(R^{L})—, —N(R^{L})C(O)O—, —S(O)—, —S(O)₂—, —S(O)(═NR^{L})—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, phenylene, 5-6 membered heteroarylene, C₃₋₇ cycloalkylene-C₁₋₄ alkyl, (4-7 membered heterocycloalkylene)-C₁₋₄ alkyl, -phenylene-C₁₋₄ alkyl-, and (5-6 membered heteroarylene)-C₁₋₄ alkyl of L¹, L², and L³ are each optionally substituted with 1, 2, 3, or 4 independently selected R^{G} substituents;
provided that when L³ is —C(O)—, —S(O)—, —S(O)₂—, or —S(O)(═NR^{L})—, then Ring C is C₉₋₁₄ cycloalkyl, C₁₀₋₁₄ aryl, 8-14 membered heterocycloalkyl, or 9-14 membered heteroaryl;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —NO₂, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}(O)NR^{c1}R^{d1}, —NR^{c1}S(O)₂R^{b1}, —NR^{c1}S(O)₂NR^{c1}R^{d1}, —NR^{c1}R^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R¹ are each optionally substituted with 1, 2, 3, 4, 5, or 6 independently selected RA substituents;
or, two R¹ together with the atoms to which they are attached form a C₃₋₇ cycloalkyl, phenyl, 4-7 membered heterocycloalkyl, or 5-6 membered heteroaryl group, wherein the C₃₋₇ cycloalkyl, phenyl, 4-7 membered heterocycloalkyl, and 5-6 membered heteroaryl group is optionally substituted with 1, 2, 3, 4, 5, or 6 independently selected RA substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1}, R^{c1}, and R^{d1} are each optionally substituted with 1, 2, 3, 4, 5, or 6 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected RA substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b1} are each optionally substituted with 1, 2, 3, 4, 5, or 6 independently selected RA substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{1A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1A}, —SR^{a1A}, —NR^{c1A}R^{d1A}, —NO₂, —C(O)R^{a1A}, —C(O)OR^{a1A}, —C(O)NR^{c1A}R^{d1A}, —C(O)NR^{c1A}(OR^{a1A}), —OC(O)R^{a1A}, —OC(O)NR^{c1A}R^{d1A}, —OC(O)OR^{a1A}, —OS(O)₂R^{b1A}, —OS(O)₂NR^{c1A}R^{d1A}, —NR^{c1A}C(O)R^{a1A}, —NR^{c1A}C(O)OR^{a1A}, —NR^{c1A}C(O)NR^{c1A}R^{d1A}, —NR^{c1A}S(O)₂R^{b1A}, —NR^{c1A}S(O)₂NR^{c1A}R^{d1A}, —NR^{c1A}OR^{a1A}, —NR^{c1A}S(O)R^{b1A}, —NR^{c1A}S(O)NR^{c1A}R^{d1A}, —S(O)R^{b1A}, —S(O)₂R^{b1A}, —S(O)NR^{c1A}R^{d1A}, —S(O)₂NR^{c1A}R^{d1A}, —C(═NR^{e1A})R^{a1A}, —C(═NR^{e1A})R^{c1A}R^{d1A}, —NR^{c1A}C(═NR^{e1A})R^{a1A}, —NR^{c1A}C(═NR^{e1A})NR^{c1A}R^{d1A}, —NR^{c1A}S(O)(═NR^{e1A})R^{b1A}—, NR^{c1A}S(O)(═NR^{e1A})NR^{clA}R^{d1A}, —OS(O)(═NR^{e1A})R^{b1A}, —S(O)(═NR^{e1A})R^{b1A}, —S(O)(═NR^{e1A})R^{c1A}R^{d1A}, —C(O)NR^{c1A}S(O)₂R^{b1A}, —C(O)NR^{c1A}S(O)₂NR^{c1A}R^{d1A}, —S(O)₂NR^{c1A}C(O)R^{b1A}, —NR^{clA}S(O)NR^{c1A}C(O)R^{b1A} and —P(O)R^{f1A}R^{g1A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{1A} are each optionally substituted with 1, 2, 3, 4, 5, or 6 independently selected R^{G} substituents;
each R^{a1A}, R^{c1A} and R^{d1A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1A}, R^{clA}, and R^{d1A} are each optionally substituted with 1, 2, 3, 4, 5, or 6 independently selected R^{G} substituents;
or, any R^{c1A} and R^{d1A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b1A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b1A} are each optionally substituted with 1, 2, 3, 4, 5, or 6 independently selected R^{G} substituents;
each R^{e1A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1A} and R^{g1A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —S(O)(═NR^{e2})R^{b2}, and —S(O)(═NR^{e2})NR^{c2}R^{d2}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R² are each optionally substituted with 1, 2, 3, 4, 5, or 6 independently selected R^{G} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a2}, R^{b2}, R^{c2}, and R^{d2} are each optionally substituted with 1, 2, 3, 4, 5, or 6 independently selected R^{G} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R³ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3}, —SR^{a3}, —NR^{c3}R^{d3}, —NO₂, —C(O)R^{a3}, —C(O)OR^{a3}, —C(O)NR^{c3}R^{d3}, —C(O)NR^{c3}(OR^{a3}), —OC(O)R^{a3}, —OC(O)NR^{c3}R^{d3}, —OC(O)OR^{a3}, —OS(O)₂R^{b3}, —OS(O)₂NR^{c3}R^{d3}, —NR^{c3}C(O)R^{a3}, —NR^{c3}C(O)OR^{a3}, —NR^{c3}C(O)NR^{c3}R^{d3}, —NR^{c3}S(O)₂R^{b3}, —NR^{c3}S(O)₂NR^{c3}R^{d3}, —NR^{c3}OR^{a3}, —NR^{c3}S(O)R^{b3}, —NR^{c3}S(O)NR^{c3}R^{d3}, —S(O)R^{b3}, —S(O)₂R^{b3}, —S(O)NR^{c3}R^{d3}, —S(O)₂NR^{c3}R^{d3}, —C(═NR^{e3})R^{a3}, —C(═NR^{e3})NR^{c3}R^{d3}, —NR^{c3}C(═NR^{e3})R^{a3}, —NR^{c3}C(═NR^{e3})NR^{c3}R^{d3}, —NR^{c3}S(O)(═NR^{e3})R^{b3}, —NR^{c3}S(O)(═NR^{e3})NR^{c3}R^{d3}, —OS(O)(═NR^{e3})R^{b3}, —S(O)(═NR^{e3})R^{b3}, —S(O)(═NR^{e3})NR^{c3}R^{d3}, —C(O)NR^{c3}S(O)₂R^{b3}, —C(O)NR^{c3}S(O)₂NR^{c3}R^{d3}, —S(O)₂NR^{c3}C(O)R^{b3}, —NR^{c3}S(O)NR^{c3}C(O)R^{b3}, and —P(O)R^{f3}R^{g3}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R³ are each optionally substituted with 1, 2, 3, 4, 5, or 6 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a3}, R^{c3}, and R^{d3} are each optionally substituted with 1, 2, 3, 4, 5, or 6 independently selected R^{3A} substituents;
or, any R^{c3} and R^{d3} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{3A} substituents;
each R^{b3} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b3} are each optionally substituted with 1, 2, 3, 4, 5, or 6 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{3A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a3A}, —SR^{a3A}, —NR^{c3A}R^{d3A}, —NO₂, —C(O)R^{a3A}, —C(O)OR^{a3A}, —C(O)NR^{c3A}R^{d3A}, —C(O)NR^{c3A}(OR^{a3A}), —OC(O)R^{a3A}, —OC(O)NR^{c3A}R^{d3A}, —OC(O)OR^{a3A}, —OS(O)₂R^{b3A}, —OS(O)₂NR^{c3A}R^{d3A}, —NR^{c3A}C(O)R^{a3A}, —NR^{c3A}C(O)OR^{a3A}, —NR^{c3A}C(O)NR^{c3A}R^{d3A}, —NR^{c3A}S(O)₂R^{b3A}, —NR^{c3A}S(O)₂NR^{c3A}R^{d3A}, —NR^{c3A}OR^{a3A}, —NR^{c3A}S(O)R^{b3A}, —NR^{c3A}S(O)NR^{c3A}R^{d3A}, —S(O)R^{b3A}, —S(O)₂R^{b3A}, —S(O)NR^{c3A}R^{d3A}, —S(O)₂NR^{c3A}R^{d3A}, —C(═NR^{e3A})R^{a3A}, —C(═NR^{e3A})NR^{c3A}R^{d3A}, —NR^{c3A}C(═NR^{e3A})R^{a3A}, —NR^{c3A}C(═NR^{e3A})NR^{c3A}R^{d3A}, —NR^{c3A}S(O)(═NR^{e3A})R^{b3A}, —NR^{c3A}S(O)(═NR^{e3A})NR^{c3A}R^{d3A}, —OS(O)(═NR^{e3A})R^{b3A}, —S(O)(═NR^{e3A})R^{b3A}, —S(O)(═NR^{e3A})NR^{c3A}R^{d3A}, —C(O)NR^{c3A}S(O)₂R^{b3A}, —C(O)NR^{c3A}S(O)₂NR^{c3A}R^{d3A}, —S(O)₂NR^{c3A}C(O)R^{b3A}, —NR^{c3A}S(O)NR^{c3A}C(O)R^{b3A} and —P(O)R^{f3A}R^{g3A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{3A} are each optionally substituted with 1, 2, 3, 4, 5, or 6 independently selected R^{G} substituents;
each R^{a3A}, R^{c3A}, and R^{d3A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a3A}, R^{c3A} and R^{d3A} are each optionally substituted with 1, 2, 3, 4, 5, or 6 independently selected R^{G} substituents;
or, any R^{c3A} and R^{d3A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b3A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b3A} are each optionally substituted with 1, 2, 3, 4, 5, or 6 independently selected R^{G} substituents;
each R^{e3A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f3A} and R^{g3A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}, —NR^{c4}R^{d4}, —C(O)R^{a4}, —C(O)OR^{a4}, —C(O)NR^{c4}R^{d4}, —C(O)NR^{c4}(OR^{a4}), —OC(O)NR^{c4}R^{d4}, —NR^{c4}C(O)R^{a4}, —NR^{c4}C(O)OR^{a4}, —NR^{c4}C(O)NR^{c4}R^{d4}, —NR^{c4}S(O)₂R^{b4}, —NR^{c4}S(O)₂NR^{c4}R^{d4}, —NR^{c4}OR^{a4}, —NR^{c4}S(O)R^{b4}, —NR^{c4}S(O)NR^{c4}R^{d4}, —S(O)R^{b4}, —S(O)₂R^{b4}, —S(O)NR^{c4}R^{d4}, —S(O)₂NR^{c4}R^{d4}, —C(═NR^{e4})R^{a4}, —C(═NR^{e4})NR^{c4}R^{d4}, —NR^{c4}C(═NR^{e4})R^{a4}, —NR^{c4}C(═NR^{e4})NR^{c4}R^{d4}, —NR^{c4}S(O)(═NR^{e4})R^{b4}, —NR^{c4}S(O)(═NR^{e4})NR^{c4}R^{d4}, —S(O)(═NR^{e4})R^{b4}, and —S(O)(═NR^{e4})NR^{c4}R^{d4}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
or, R⁴ and L¹, together with the atoms to which they are attached, form a C₅₋₁₀ cycloalkyl, phenyl, 5-10 membered heterocycloalkyl, or 5-6 membered heteroaryl group, wherein the C₅₋₁₀ cycloalkyl, phenyl, 5-10 membered heterocycloalkyl, and 5-6 membered heteroaryl group is optionally substituted with 1, 2, 3, 4, 5, or 6 independently selected R^{G} substituents;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a4}, R^{b4}, R^{c4}, and R^{d4} are each optionally substituted with 1, 2, 3, 4, 5, or 6 independently selected R^{G} substituents;
or, any R^{c4} and R^{d4} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, 4, 5, or 6 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁-4 alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5}, R^{c5}, and R^{d5} are each optionally substituted with 1, 2, 3, 4, 5, or 6 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b5} are each optionally substituted with 1, 2, 3, 4, 5, or 6 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{5A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5A}, —SR^{a5A}, —NR^{c5A}R^{d5A}, —NO₂—C(O)R^{a5A}, —C(O)OR^{a5A}, —C(O)NR^{c5A}R^{d5A}, —C(O)NR^{c5A}(OR^{a5A}), —OC(O)R^{a5}, —OC(O)NR^{c5A}R^{d5A}, —OC(O)OR^{a5A}, —OS(O)₂R^{b5A}, —OS(O)₂NR^{c5A}R^{d5A}, —NR^{c5A}C(O)R^{a5A}, —NR^{c5A}C(O)OR^{a5A}, —NR^{c5A}C(O)NR^{c5A}R^{d5A}, —NR^{c5A}S(O)₂R^{b5A}, —NR^{c5A}S(O)₂NR^{c5A}R^{d5A}, —NR^{c5A}OR^{a5A}, —NR^{c5A}S(O)R^{b5A}, —NR^{c5A}S(O)NR^{c5A}R^{d5A}, —S(O)R^{b5A}, —S(O)₂R^{b5A}, —S(O)NR^{c5A}R^{d5A}, —S(O)₂NR^{c5A}R^{d5A}, —C(═NR^{e5A})R^{a5A}, —C(═NR^{e5A})NR^{c5A}R^{d5A}, —NR^{c5A}C(═NR^{e5A})R^{a5A}, —NR^{c5A}C(═NR^{e5A})NR^{c5A}R^{d5A}, —NR^{c5A}S(O)(═NR^{e5A})R^{b5A}, —NR^{c5A}S(O)(═NR^{e5A})NR^{c5A}R^{d5A}, —OS(O)(═NR^{e5A})R^{b5A}, —S(O)(═NR^{e5A})R^{b5A}, —S(O)(═NR^{e5A})NR^{c5A}R^{d5A}, —C(O)NR^{c5A}S(O)₂R^{b5A}, —C(O)NR^{c5A}S(O)₂NR^{c5A}R^{d5A}, —S(O)₂NR^{c5A}C(O)R^{b5A}, —NR^{c5A}S(O)NR^{c5A}C(O)R^{b5A} and —P(O)R^{f5A}R^{g5A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{5A} are each optionally substituted with 1, 2, 3, 4, 5, or 6 independently selected R^{G} substituents;
each R^{a5A}, R^{c5A}, and R^{d5A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5A}, R^{c5A} and R^{d5A} are each optionally substituted with 1, 2, 3, 4, 5, or 6 independently selected R^{G} substituents;
or, any R^{c5A} and R^{d5A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b5A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b5A} are each optionally substituted with 1, 2, 3, 4, 5, or 6 independently selected R^{G} substituents;
each R^{e5A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5A} and R^{g5A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl;
R⁹ is selected from H, halo, CN, C₁₋₆ alkyl, or C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein X¹ is N.

3. The compound of claim 1 or 2, or a pharmaceutically acceptable salt thereof, wherein X² is N.

4. The compound of any one of claims 1 to 3, or a pharmaceutically acceptable salt thereof, wherein X³ is N.

5. The compound of any one of claims 1 to 4, or a pharmaceutically acceptable salt thereof, wherein X⁴ is C.

6. The compound of any one of claims 1 to 5, or a pharmaceutically acceptable salt thereof, wherein Y is N.

7. The compound of any one of claims 1 to 6, or a pharmaceutically acceptable salt thereof, wherein Z is C.

8. The compound of any one of claims 1 to 7, or a pharmaceutically acceptable salt thereof, wherein L¹ is selected from a bond, C₁₋₆ alkylene, and C₁₋₆ haloalkylene.

9. The compound of any one of claims 1 to 7, or a pharmaceutically acceptable salt thereof, wherein L¹ is C₁₋₆ alkylene.

10. The compound of any one of claims 1 to 7, or a pharmaceutically acceptable salt thereof, wherein L¹ is —CH₂—.

11. The compound of any one of claims 1 to 10, or a pharmaceutically acceptable salt thereof, wherein L² is selected from —N(R^{L})—, —C(O)—, and —N(R^{L})C(O)—.

12. The compound of any one of claims 1 to 10, or a pharmaceutically acceptable salt thereof, wherein L² is —N(R^{L})C(O)—.

13. The compound of any one of claims 1 to 12, or a pharmaceutically acceptable salt thereof, wherein each R^{L} is independently selected from H and C₁₋₆ alkyl.

14. The compound of any one of claims 1 to 10, or a pharmaceutically acceptable salt thereof, wherein L² is —NHC(O)—.

15. The compound of any one of claims 1 to 14, or a pharmaceutically acceptable salt thereof, wherein L³ is C₁₋₆ alkylene or —C(O)—.

16. The compound of any one of claims 1 to 14, or a pharmaceutically acceptable salt thereof, wherein L³ is —CH₂— or —C(O)—.

17. The compound of any one of claims 1 to 16, or a pharmaceutically acceptable salt thereof, wherein Ring A is C₃₋₇ cycloalkyl, phenyl, 4-7 membered heterocycloalkyl, or 5-6 membered heteroaryl.

18. The compound of any one of claims 1 to 16, or a pharmaceutically acceptable salt thereof, wherein Ring A is phenyl.

19. The compound of any one of claims 1 to 18, or a pharmaceutically acceptable salt thereof, wherein n is 1 or 2.

20. The compound of any one of claims 1 to 19, or a pharmaceutically acceptable salt thereof, wherein each R¹ is independently selected from halo, C₁₋₆ alkyl, C₂₋₆ alkenyl, C₂₋₆ alkynyl, and C₁₋₆ haloalkyl.

21. The compound of any one of claims 1 to 19, or a pharmaceutically acceptable salt thereof, wherein each R¹ is independently selected from halo, C₁₋₆ alkyl, and C₁₋₆ haloalkyl.

22. The compound of any one of claims 1 to 19, or a pharmaceutically acceptable salt thereof, wherein each R¹ is independently selected from halo and C₁₋₆ haloalkyl.

23. The compound of any one of claims 1 to 19, or a pharmaceutically acceptable salt thereof, wherein each R¹ is independently selected from chloro and trifluoromethyl.

24. The compound of any one of claims 1 to 16, or a pharmaceutically acceptable salt thereof, wherein Ring A is chloro(trifluoromethyl)phenyl.

25. The compound of any one of claims 1 to 24, or a pharmaceutically acceptable salt thereof, wherein Ring B is C₃₋₇ cycloalkyl, phenyl, 4-7 membered heterocycloalkyl, or 5-6 membered heteroaryl.

26. The compound of any one of claims 1 to 24, or a pharmaceutically acceptable salt thereof, wherein Ring B is 4-7 membered heterocycloalkyl.

27. The compound of any one of claims 1 to 24, or a pharmaceutically acceptable salt thereof, wherein Ring B is piperazinyl.

28. The compound of any one of claims 1 to 27, or a pharmaceutically acceptable salt thereof, wherein m is 0.

29. The compound of any one of claims 1 to 28, or a pharmaceutically acceptable salt thereof, wherein Ring C is 8-14 membered heterocycloalkyl, 5-6 membered heteroaryl, or 9-14 membered heteroaryl.

30. The compound of any one of claims 1 to 28, or a pharmaceutically acceptable salt thereof, wherein Ring C is 8-14 membered heterocycloalkyl or 9-14 membered heteroaryl.

31. The compound of any one of claims 1 to 28, or a pharmaceutically acceptable salt thereof, wherein Ring C is 9-14 membered heteroaryl.

32. The compound of any one of claims 1 to 28, or a pharmaceutically acceptable salt thereof, wherein Ring C is pyridinyl, 5H-pyrrolo[3,2-d]pyrimidinyl, quinolinyl, or isoquinolinyl.

33. The compound of any one of claims 1 to 32, or a pharmaceutically acceptable salt thereof, wherein p is 0, 1, or 2.

34. The compound of any one of claims 1 to 33, or a pharmaceutically acceptable salt thereof, wherein each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3}; and each R^{a3}, R^{c3}, and R^{d3} is independently selected from H, C₁₋₆ alkyl, and C₁₋₆ haloalkyl.

35. The compound of any one of claims 1 to 33, or a pharmaceutically acceptable salt thereof, wherein each R³ is independently selected from —OR^{a3}; and each R^{a3}, R^{c3}, and R^{d3} is independently selected from H and C₁₋₆ alkyl.

36. The compound of any one of claims 1 to 33, or a pharmaceutically acceptable salt thereof, wherein each R³ is hydroxy.

37. The compound of any one of claims 1 to 36, or a pharmaceutically acceptable salt thereof, wherein R⁴ is selected from H, halo, C₁₋₆ alkyl, C₂₋₆ alkenyl, C₂₋₆ alkynyl, C₁₋₆ haloalkyl.

38. The compound of any one of claims 1 to 36, or a pharmaceutically acceptable salt thereof, wherein R⁴ is C₁₋₆ alkyl.

39. The compound of any one of claims 1 to 36, or a pharmaceutically acceptable salt thereof, wherein R⁴ is ethyl.

40. The compound of any one of claims 1 to 39, or a pharmaceutically acceptable salt thereof, wherein R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents.

41. The compound of any one of claims 1 to 39, or a pharmaceutically acceptable salt thereof, wherein R⁵ is selected from C₃₋₁₀ cycloalkyl, C₆₋₁₀ aryl, 4-10 membered heterocycloalkyl, and 5-10 membered heteroaryl, wherein the C₃₋₁₀ cycloalkyl, C₆₋₁₀ aryl, 4-10 membered heterocycloalkyl, and 5-10 membered heteroaryl of R⁵ are each optionally substituted with 1, 2, 3, or 4 independently selected R^{5A} substituents.

42. The compound of any one of claims 1 to 39, or a pharmaceutically acceptable salt thereof, wherein R⁵ is phenyl or 4-10 membered heterocycloalkyl, wherein the phenyl and 4-10 membered heterocycloalkyl of R⁵ are each optionally substituted with 1, 2, 3, or 4 independently selected R^{5A} substituents;
each R^{5A} is independently selected from —S(O)R^{b5A}, —S(O)₂R^{b5A}, —S(O)NR^{c5A}R^{d5A}, —S(O)₂NR^{c5A}R^{d5A} and —P(O)R^{f5A}R^{g5A}; and
each R^{a5A}, R^{b5A}, R^{c5A}, R^{d5A}, R^{f5A}, and R^{g5A} is independently selected from H, C₁₋₆ alkyl, C₁₋₆ haloalkyl, C₂₋₆ alkenyl, C₂₋₆ alkynyl.

43. The compound of any one of claims 1 to 39, or a pharmaceutically acceptable salt thereof, wherein R⁵ is phenyl, which is optionally substituted with —S(O)R^{b5A} or —P(O)R^{f5A}R^{g5A}, wherein R^{b5A}, R^{f5A}, and R^{g5A} are each independently C₁₋₆ alkyl.

44. The compound of any one of claims 1 to 39, or a pharmaceutically acceptable salt thereof, wherein R⁵ is 4-7 membered heterocycloalkyl.

45. The compound of any one of claims 1 to 39, or a pharmaceutically acceptable salt thereof, wherein R⁵ is phenyl, dihydropyranyl, or oxaazaspiro[4.5]decanyl, wherein the phenyl is optionally substituted with methylsulfonyl or dimethylphosphoryl.

46. The compound of claim 1, or a pharmaceutically acceptable salt thereof, wherein:
X¹ is N, NR⁶, or CR⁷;
X² is N, NR⁸, or CR⁹;
X³ is C or N;
X⁴ is C or N;
Y is C or N;
Z is C or N;
each [symbol] is independently a single or double bond;
n is 0, 1, 2, or 3;
m is 0, 1, or 2;
p is 0, 1, 2, or 3;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is C₁₋₆ alkylene, —C(O)—, —S(O)—, —S(O)₂—, or —S(O)(═NR^{L});
provided that when L³ is —C(O)—, —S(O)—, —S(O)₂—, or —S(O)(═NR^{L})—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents;
each R^{5A} is independently selected from —S(O)R^{b5A}, —S(O)₂R^{b5A}, —S(O)NR^{c5A}R^{d5A}, —S(O)₂NR^{c5A}R^{d5A} and —P(O)R^{f5A}R^{g5A};
each R^{a5A}, R^{b5A}, R^{c5A}, R^{d5A}, R^{f5A}, and R^{g5A} is independently selected from H, C₁₋₆ alkyl, C₁₋₆ haloalkyl, C₂₋₆ alkenyl, C₂₋₆ alkynyl;
R⁶ is selected from H, C₁₋₆ alkyl, or C₁₋₆ haloalkyl;
R⁷ is selected from H, halo, CN, C₁₋₆ alkyl, or C₁₋₆ haloalkyl;
R⁸ is selected from H, C₁₋₆ alkyl, or C₁₋₆ haloalkyl; and
R⁹ is selected from H, halo, CN, C₁₋₆ alkyl, or C₁₋₆ haloalkyl.

47. The compound of claim 1, or a pharmaceutically acceptable salt thereof, wherein:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is selected from a bond, C₁₋₆ alkylene, and C₁₋₆ haloalkylene;
L² is selected from —N(R^{L})—, —C(O)—, and —N(R^{L})C(O)—;
R^{L} is selected from H and C₁₋₆ alkyl;
L³ is C₁₋₆ alkylene, —C(O)—, —S(O)—, —S(O)₂—, or —S(O)(═NR^{L});
provided that when L³ is —C(O)—, —S(O)—, —S(O)₂—, or —S(O)(═NR^{L})—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁵ are each optionally substituted with 1, 2, 3, or 4 independently selected R^{5A} substituents;
each R^{5A} is independently selected from —S(O)R^{b5A}, S(O)₂R^{b5A}, and —P(O)R^{f5A}R^{g5A}; and
each R^{b5A}, R^{f5A}, and R^{g5A} is independently selected from H and C₁₋₆ alkyl.

48. The compound of claim 1, or a pharmaceutically acceptable salt thereof, wherein:
X¹, X², and X³ are each N;
X⁴ is C;
Y is N;
Z is C;
n is 1 or 2;
m is 0;
p is 0, 1, or 2;
Ring A is C₃₋₇ cycloalkyl, phenyl, 4-7 membered heterocycloalkyl, or 5-6 membered heteroaryl;
Ring B is C₃₋₇ cycloalkyl, phenyl, 4-7 membered heterocycloalkyl, or 5-6 membered heteroaryl;
Ring C is 8-14 membered heterocycloalkyl, 5-6 membered heteroaryl, or 9-14 membered heteroaryl;
L¹ is —CH₂—;
L² is —NHC(O)—;
L³ is —CH₂— or —C(O)—;
provided that when L³ is —C(O)—, then Ring C is 8-14 membered heterocycloalkyl or 9-14 membered heteroaryl;
each R¹ is independently selected from halo, C₁₋₆ alkyl, and C₁₋₆ haloalkyl;
each R³ is independently selected from H, halo, C₁₋₆ alkyl, C₂₋₆ alkenyl, C₂₋₆ alkynyl, C₁₋₆ haloalkyl, —CN, —OR^{a3}, —SR^{a3}, and —NR^{c3}R^{d3};
each R^{a3}, R^{c3}, and R^{d3} is independently selected from H, C₁₋₆ alkyl, and C₁₋₆ haloalkyl;
R⁴ is selected from H, halo, C₁₋₆ alkyl, C₂₋₆ alkenyl, C₂₋₆ alkynyl, C₁₋₆ haloalkyl;
R⁵ is selected from C₃₋₁₀ cycloalkyl, C₆₋₁₀ aryl, 4-10 membered heterocycloalkyl, and 5-10 membered heteroaryl, wherein the C₃₋₁₀ cycloalkyl, C₆₋₁₀ aryl, 4-10 membered heterocycloalkyl, and 5-10 membered heteroaryl of R⁵ are each optionally substituted with 1, 2, 3, or 4 independently selected R^{5A} substituents;
each R^{5A} is independently selected from —S(O)R^{b5A}, S(O)₂R^{b5A}, and —P(O)R^{f5A}R^{g5A}; and
each R^{b5A}, R^{f5A}, and R^{g5A} is independently selected from H and C₁₋₆ alkyl.

49. The compound of claim 1, wherein the compound of Formula I is a compound of Formula II: 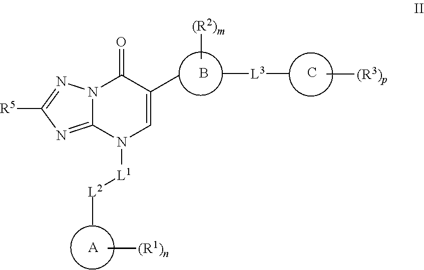
or a pharmaceutically acceptable salt thereof.

50. The compound of claim 1, wherein the compound of Formula I is a compound of Formula III: 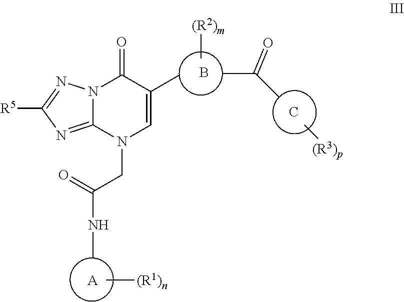
or a pharmaceutically acceptable salt thereof.

51. The compound of claim 1, wherein the compound of Formula I is a compound of Formula IV: 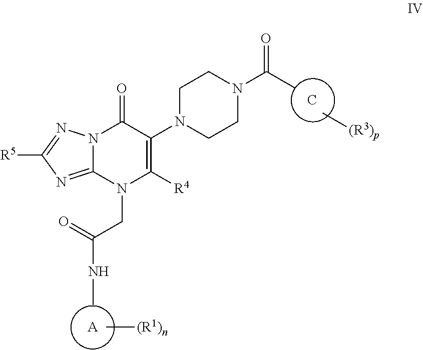
or a pharmaceutically acceptable salt thereof.

52. The compound of claim 1, wherein the compound of Formula I is a compound of Formula V: 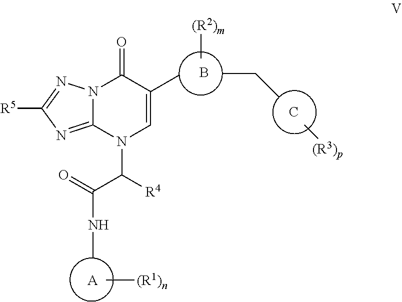
or a pharmaceutically acceptable salt thereof.

53. The compound of claim 1, wherein the compound of Formula I is a compound of Formula VI: 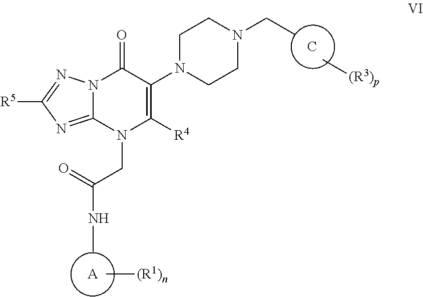
or a pharmaceutically acceptable salt thereof.

54. The compound of claim 1, which is selected from:
N-[2-chloro-4-(trifluoromethyl)phenyl]-2-[2-(3,6-dihydro-2H-pyran-4-yl)-5-ethyl-6-[4-(4-hydroxyisoquinoline-3-carbonyl)piperazin-1-yl]-7-oxo-[1,2,4]triazolo[1,5-a]pyrimidin-4-yl]acetamide;
N-[2-chloro-4-(trifluoromethyl)phenyl]-2-[2-(3,6-dihydro-2H-pyran-4-yl)-5-ethyl-6-[4-(3-hydroxyquinoline-2-carbonyl)piperazin-1-yl]-7-oxo-[1,2,4]triazolo[1,5-a]pyrimidin-4-yl]acetamide; and
N-[2-chloro-4-(trifluoromethyl)phenyl]-2-[2-(3,6-dihydro-2H-pyran-4-yl)-5-ethyl-7-oxo-6-[4-(5H-pyrrolo[3,2-d]pyrimidine-4-carbonyl)piperazin-1-yl]-[1,2,4]triazolo[1,5-a]pyrimidin-4-yl]acetamide;
or a pharmaceutically acceptable salt thereof.

55. The compound of claim 1, which is selected from:
N-[2-chloro-4-(trifluoromethyl)phenyl]-2-[2-(3,6-dihydro-2H-pyran-4-yl)-5-ethyl-6-[4-[(3-hydroxy-2-pyridyl)methyl]piperazin-1-yl]-7-oxo-[1,2,4]triazolo[1,5-a]pyrimidin-4-yl]acetamide;
N-[2-chloro-4-(trifluoromethyl)phenyl]-2-[2-(3,6-dihydro-2H-pyran-4-yl)-5-ethyl-6-[4-(7-hydroxyquinoline-8-carbonyl)piperazin-1-yl]-7-oxo-[1,2,4]triazolo[1,5-a]pyrimidin-4-yl]acetamide;
N-(2-chloro-4-(trifluoromethyl)phenyl)-2-(5-ethyl-6-(4-(7-hydroxyquinoline-8-carbonyl)piperazin-1-yl)-7-oxo-2-(1-oxa-8-azaspiro[4.5]decan-8-yl)-[1,2,4]triazolo[1,5-a]pyrimidin-4(7H)-yl)acetamide;
N-(2-chloro-4-(trifluoromethyl)phenyl)-2-(5-ethyl-6-(4-((3-hydroxypyridin-2-yl)methyl)piperazin-1-yl)-7-oxo-2-(1-oxa-8-azaspiro[4.5]decan-8-yl)-[1,2,4]triazolo[1,5-a]pyrimidin-4(7H)-yl)acetamide; and
N-(2-chloro-4-(trifluoromethyl)phenyl)-2-(5-ethyl-6-(4-(7-hydroxyquinoline-8-carbonyl)piperazin-1-yl)-2-(4-(methylsulfonyl)phenyl)-7-oxo-[1,2,4]triazolo[1,5-a]pyrimidin-4(7H)-yl)acetamide;
or a pharmaceutically acceptable salt thereof.

56. The compound of claim 1, which is selected from:
N-(2-chloro-4-(trifluoromethyl)phenyl)-2-(5-ethyl-6-(4-((3-hydroxypyridin-2-yl)methyl)piperazin-1-yl)-2-(4-(methylsulfonyl)phenyl)-7-oxo-[1,2,4]triazolo[1,5-a]pyrimidin-4(7H)-yl)acetamide; and
N-(2-chloro-4-(trifluoromethyl)phenyl)-2-(2-(4-(dimethylphosphoryl)phenyl)-5-ethyl-6-(4-((3-hydroxypyridin-2-yl)methyl)piperazin-1-yl)-7-oxo-[1,2,4]triazolo[1,5-a]pyrimidin-4(7H)-yl)acetamide;
or a pharmaceutically acceptable salt thereof.

57. A pharmaceutical composition, comprising a compound of any one of claims 1 to 56, or a pharmaceutically acceptable salt thereof, and a pharmaceutically acceptable carrier.

58. A method of inhibiting an activity of a WRN protein, comprising contacting the protein with a compound of any one of claims 1 to 56, or a pharmaceutically acceptable salt thereof.

59. A method of treating a WRN-mediated disease or disorder in a patient, comprising administering to the patient a therapeutically effective amount of a compound of any one of claims 1 to 56, or a pharmaceutically acceptable salt thereof.

60. The method of claim 59, wherein the disease or disorder is a cancer.

61. The method of claim 60, wherein the cancer is characterized as exhibiting defective DNA mismatch repair (dMMR).

62. The method of claim 60, wherein the cancer is characterized as exhibiting microsatellite instability-high (MSI-H).

63. The method of any one of claims 59 to 62, wherein the disease or disorder is selected from colon cancer, small intestine cancer, endometrial cancer, gastric cancer, ovarian cancer, pancreatic cancer, cholangiocarcinoma, rectal cancer, adrenal cancer, breast cancer, uterine cancer, cervical cancer, Wilms tumor, mesothelioma, head and neck cancer, esophageal cancer, lung cancer, kidney cancer, sarcoma cancer, liver cancer, melanoma, prostate cancer, bladder cancer, glioblastoma, and neuroendocrine cancer.
